(19) United States
(12) Patent Application Publication
(10) Pub. No.: US 2012/0022107 A1
(43) Pub. Date: Jan. 26, 2012

(54) 2-(PIPERIDIN-1-YL)-4-AZOLYL-THIAZOLE-5-CARBOXYLIC ACID DERIVATIVES AGAINST BACTERIAL INFECTIONS

(75) Inventors: Shahul Hameed Peer Mohamed, Bangalore (IN); David Waterson, Cheshire (GB)

(73) Assignee: ASTRAZENECA AB, Sodertalje (SE)

(21) Appl. No.: 13/139,079

(22) PCT Filed: Dec. 11, 2009
(86) PCT No.: PCT/GB09/51695

§ 371 Date: Oct. 12, 2011

Related U.S. Application Data
(60) Provisional application No. 61/121,947, filed on Dec. 12, 2008.

(51) Int. Cl. A61K 31/454; A61P 31/04; C07D 417/14

(52) U.S. Cl. 514/326; 546/209

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

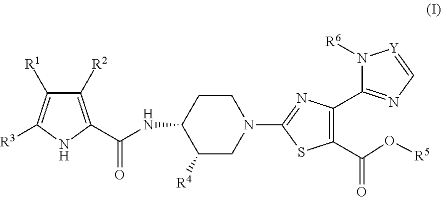

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae, multiple resistant Enterococcus faecium and multi drug resistant Mycobacterium tuberculosis (MDR and XDR TB).

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT, interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomeraseIV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087). AstraZeneca has also published certain applications describing anti-bacterial compounds, in particular WO2006/087543.

We have discovered a new class of compounds which are useful for inhibiting DNA gyrase and/or topoisomerase IV.

According to the present invention there is provided a compound of formula (I):

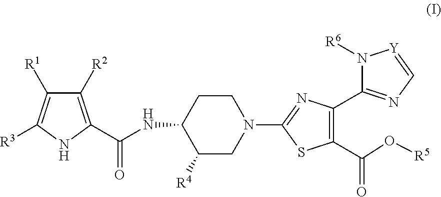

wherein:
(i) R¹ is Cl, R² is Br or CF₃ and R³ is CH₃,
(ii) R¹ is Br, R² is Cl, Br, CN or CF₃ and R³ is CH₃,
(iii) R¹ is CN, R² is Br or CF₃ and R³ is CH₃, or
(iv) R¹ is CH₃, R² and R³ are Cl;
R⁴ is H, fluoro, methyl, methoxy, ethoxy, cyclopropylmethoxy, propoxy, allyloxy and benzyloxy;
R⁵ is hydrogen or C₁₋₄alkyl;
Y=N or C—R^{a} wherein R^{a} is H, CH₃, F, CF₃, or CN;
R⁶ is selected from any one of C₁₋₄alkyl, C₁₋₄ haloalkyl C₂₋₄alkenyl, C₂₋₄alkenyl, C₃₋₆cycloalkyl, (C₃₋₆cycloalkyl)alkyl, (C₁₋₄alkoxy)alkyl, (C₃₋₆ cycloalkoxy)alkyl, (C₁₋₄ haloalkoxy)alkyl, C₁₋₄alkanoyl, N—(C₁₋₄alkyl)alkyl, N,N—(C₁₋₄alkyl)₂alkyl, carbocyclyl-R⁷— or heterocyclyl-R⁸—
R⁷ and R⁸ are independently selected from a direct bond, —O—, —N(R⁹)—, —C(O)—, —N(R¹⁰)C(O)—, —C(O)N(R¹¹)—, —S(O)ₚ—, —SO₂N(R¹²)— or —N(R¹³)SO₂—; wherein R⁹, R¹⁰, R¹¹, R¹² and R¹³ are independently selected from hydrogen or C₁₋₄alkyl and p is 0-2;

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

An example of “(C₁₋₄alkoxy)alkyl” is methoxy ethyl. Examples of “C₁₋₄alkoxycarbonyl” are methoxycarbonyl, ethoxycarbonyl, n- and t-butoxycarbonyl. Examples of “(C₁₋₄alkoxy)alkyl” are methoxy ethyl and isopropoxy ethyl. Examples of “C₁₋₄alkanoyl” are propionyl and acetyl. Examples of “C₂₋₄alkenyl” are vinyl, allyl and 1-propenyl. Examples of “C₂₋₄alkynyl” are ethynyl, 1-propynyl and 2-propynyl. Examples of “C₁₋₄ halo alkyl” are trifluoromethyl and 2,2-difluoroethyl. Examples of “C₃₋₆cycloalkyl” are cyclopropyl and cyclopenty. Examples of “(C₃₋₆cycloalkyl)alky” are cyclopropyl methyl and cyclopentymethyl. Examples of “(C₃₋₆cycloalkyl)alky” are cyclopropyl methyl and cyclopentymethyl. Examples of “(C₃₋₆cycloalkoxy)alky” are cyclopropyloxy ethyl and cyclopentyloxyethy. Examples of “(C₁₋₄ halo alkoxy)alkyl” are trifluoromethoxy ethyl and difluoromethoxy ethyl. Examples of “N—(C₁₋₄alkyl)alkylalkyl” are methylaminoethyl and isopropylaminoethyl. Examples of “N,N—(C₁₋₄alkyl)₂alky” are N,N,dimethylamino ethyl.

The term “heterocyclyl” denotes a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

The term “carbocyclyl” denotes a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. In one aspect of the invention the pharmaceutically-acceptable salt is the sodium salt.

However, to facilitate isolation of the salt during preparation, salts which are less soluble in the chosen solvent may be utilised whether pharmaceutically-acceptable or not.

Within the present invention it is to be understood that a compound of the formula (I)) or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that in addition to the two asymmetric carbons drawn in formula (I) compounds of formula (I) may contain additional asymmetrically substituted carbon(s) and sulphur atom(s), and accordingly may exist in, and be isolated in, as far as those additional asymmetrically substituted carbon(s) and sulphur atom(s) are concerned, optically-active and racemic forms at those positions. It is to be understood that the present invention encompasses any racemic, optically-active, polymorphic or stereoisomeric form, or mixtures thereof, at any additional asymmetrically substituted carbon(s) and sulphur atom(s), which possesses properties useful in the inhibition of DNA gyrase and/or topoisomerase IV.

Optically-active forms may be prepared by procedures known in the art for example, by resolution of the racemic form by recrystallization techniques, by synthesis from optically-active starting materials, by chiral synthesis, by enzymatic resolution, by biotransformation, or by chromatographic separation using a chiral stationary phase.

Some compounds may exhibit polymorphism. It is to be understood that the present invention encompasses any polymorphic form, or mixtures thereof, which form possesses properties useful in the inhibition of DNA gyrase and/or topoisomerase IV

There follow particular and suitable values for certain substituents and groups referred to in this specification. These values may be used where appropriate with any of the definitions and embodiments disclosed hereinbefore, or hereinafter. For the avoidance of doubt each stated value for each substituent or any combination of values represents a particular and independent aspect of this invention.

It is also to be understood that certain compounds of the formula (I) and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

### R¹, R², and R³

In one aspect, R¹, R² and R³ are conveniently selected from any of the following combinations:
a) R¹ is Cl, R² is Br or CF₃ and R³ is CH₃;
b) R¹ is Br, R² is Cl, Br, CN or CF₃ and R³ is CH₃;
c) R¹ is CN, R² is Br or CF₃ and R³ is CH₃; and
d) R¹ is CH₃, then R² and R³ are Cl.

In another aspect, R¹, R² and R³ are conveniently selected from any of the following combinations:
a) R¹ is Cl, R² is Br, and R³ is CH₃;
b) R¹ is Br, R² is Cl or CN;
c) R¹ is CN, R² is Br, and R³ is CH₃; and
d) R¹ is CH₃, and R² and R³ are Cl.

### R⁴

In one aspect, R⁴ is selected from any one of H, F, CH₃, OCH₃, OCH₂CH₃, OCH₂CH₂═CH₂,

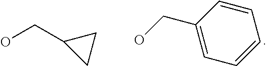

In another aspect, R⁴ is selected from fluoro, methoxy, ethoxy, and cyclopropylmethoxy.

### R⁵

In one aspect, R⁵ is selected from any one of H, CH₃, CH₂CH₃, CH₂CH₂CH₃, CH(CH₃)₂, C(CH₃)₃.

In another aspect, R⁵ is H.

### Y

In one aspect, Y is N or C—R^{a} wherein R^{a} is selected from any one of H, CH₃, F, CF₃, and CN.

In another aspect, Y is selected from CH and N.

### R⁶

In one aspect, R⁶ is a substituent on nitrogen and is selected from any one of H, CH₃, CH₂CH₃, CH₂CF₃, CH₂OCH₃, CH₂CH₂OCH₃, CH₂CH₂OCF₃, CH₂OCH₂CF₃,

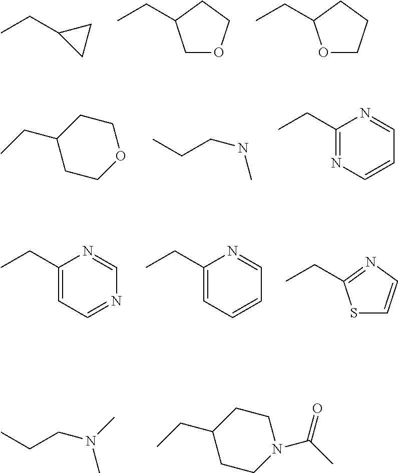

In another aspect, R⁶ is selected from C₁₋₄alkyl, (C₁₋₄alkoxy)alkyl, and (C₃₋₆cycloalkyl)alkyl.

In another aspect, R⁶ is selected from cyclopropylmethyl, ethyl, methyl, and methoxyethyl.

### R⁷ and R⁸

In one aspect, R⁷ and R⁸, are independently selected from a direct bond, —O—, —N(R⁹)—, —C(O)—, —N(R¹⁹)C(O)—, —C(O)N(R¹¹)—, —S(O)ₚ—, —SO₂N(R¹²)— or —N(R¹³)SO₂—; wherein R⁹, R¹⁰, R¹¹, R¹² and R¹³ are independently selected from hydrogen or C₁₋₄alkyl and p is 0-2.

### R¹, R², R³, R⁴, R⁵, Y, R⁶

In one aspect, R¹, R² and R³ are conveniently selected from any of the following combinations:
a) R¹ is Cl, R² is Br, and R³ is CH₃;
b) R¹ is Br, R² is Cl or CN;
c) R¹ is CN, R² is Br, and R³ is CH₃; and
d) R¹ is CH₃, and R² and R³ are Cl;
R⁴ is selected from fluoro, methoxy, ethoxy, and cyclopropylmethoxy;
Y is selected from CH and N; and
R⁶ is selected from C₁₋₄alkyl, (C₁₋₄alkoxy)alkyl, and (C₃₋₆cycloalkyl)alkyl.

Particular compounds of the invention are the compounds of the Examples, each of which provides a further independent aspect of the invention. In further aspects, the present invention also comprises any two or more compounds of the Examples.

In one aspect, the present invention provides a compound selected from:
2-((3S,4R)-4-{[3-Bromo-4-chloro-5-methyl-1H-pyrrole-2-carbonyl]amino}-3-methoxypiperidin-1-yl)-4-(1-methyl-1H-1,2,4-triazol-5-yl)-1,3-thiazole-5-carboxylic acid;
2-((3S,4R)-4-{[4-Bromo-3-chloro-5-methyl-1H-pyrrole-2-carbonyl]amino}-3-methoxypiperidin-1-yl)-4-(1-methyl-1H-1,2,4-triazol-5-yl)-1,3-thiazole-5-carboxylic acid;
2-((3S,4R)-4-{[(3-bromo-4-chloro-5-methyl-1H-pyrrol-2-yl)carbonyl]amino}-3-methoxypiperidin-1-yl)-4-(1-methyl-1H-imidazol-2-yl)-1,3-thiazole-5-carboxylic acid;
2-((3S,4R)-4-{[(3,5-Dichloro-4-methyl-1H-pyrrol-2-yl)carbonyl]amino}-3-methoxypiperidin-1-yl)-4-(1-methyl-1H-1,2,4-triazol-5-yl)-1,3-thiazole-5-carboxylic acid;
2-((3S,4R)-4-{[(3-Bromo-4-chloro-5-methyl-1H-pyrrol-2-yl)carbonyl]amino}-3-fluoropiperidin-1-yl)-4-(1-methyl-1H-1,2,4-triazol-5-yl)-1,3-thiazole-5-carboxylic acid;
2-((3S,4R)-4-{[(4-Bromo-3-chloro-5-methyl-1H-pyrrol-2-yl)carbonyl]amino}-3-fluoropiperidin-1-yl)-4-(1-methyl-1H-1,2,4-triazol-5-yl)-1,3-thiazole-5-carboxylic acid;
2-((3S,4R)-4-{[(3-Bromo-4-chloro-5-methyl-1H-pyrrol-2-yl)carbonyl]amino}-3-methoxypiperidin-1-yl)-4-[1-(2-methoxyethyl)-1H-imidazol-2-yl]-1,3-thiazole-5-carboxylic acid;
2-{(3S,4R)-4-[(3-Bromo-4-cyano-5-methyl-1H-pyrrole-2-carbonyl)-amino]-3-methoxy-piperidin-1-yl}-4-(2-methyl-2H-[1,2,4]triazol-3-yl)-thiazole-5-carboxylic acid;
2-{(3S,4R)-4-[(4-Bromo-3-cyano-5-methyl-1H-pyrrole-2-carbonyl)-amino]-3-methoxy-piperidin-1-yl}-4-(2-methyl-2H-[1,2,4]triazol-3-yl)-thiazole-5-carboxylic acid;
2-((3S,4R)-4-(3-bromo-4-chloro-5-methyl-1H-pyrrole-2-carboxamido)-3-ethoxypiperidin-1-yl)-4-(1-methyl-1H-1,2,4-triazol-5-yl)thiazole-5-carboxylic acid;
2-((3S,4R)-4-(4-bromo-3-chloro-5-methyl-1H-pyrrole-2-carboxamido)-3-ethoxypiperidin-1-yl)-4-(1-methyl-1H-1,2,4-triazol-5-yl)thiazole-5-carboxylic acid;
2-((3S,4R)-4-(3-bromo-4-chloro-5-methyl-1H-pyrrole-2-carboxamido)-3-(cyclopropylmethoxy)piperidin-1-yl)-4-(1-methyl-1H-1,2,4-triazol-5-yl)thiazole-5-carboxylic acid;
2-((3S,4R)-4-(3-bromo-4-chloro-5-methyl-1H-pyrrole-2-carboxamido)-3-methoxypiperidin-1-yl)-4-(1-ethyl-1H-1,2,4-triazol-5-yl)thiazole-5-carboxylic acid;
2-((3S,4R)-4-(4-bromo-3-chloro-5-methyl-1H-pyrrole-2-carboxamido)-3-methoxypiperidin-1-yl)-4-(1-ethyl-1H-1,2,4-triazol-5-yl)thiazole-5-carboxylic acid;
2-{(3S,4R)-4-[(3,5-Dichloro-4-methyl-1H-pyrrole-2-carbonyl)-amino]-3-methoxy-piperidin-1-yl}-4-(2-ethyl-2H-[1,2,4]triazol-3-yl)-thiazole-5-carboxylic acid;
2-((3S,4R)-4-(3-bromo-4-chloro-5-methyl-1H-pyrrole-2-carboxamido)-3-methoxypiperidin-1-yl)-4-(1-(cyclopropylmethyl)-1H-1,2,4-triazol-5-yl)thiazole-5-carboxylic acid;
2-((3S,4R)-4-(3-bromo-4-chloro-5-methyl-1H-pyrrole-2-carboxamido)-3-fluoropiperidin-1-yl)-4-(1-(cyclopropylmethyl)-1H-1,2,4-triazol-5-yl)thiazole-5-carboxylic acid;
2-((3S,4R)-4-(4-bromo-3-chloro-5-methyl-1H-pyrrole-2-carboxamido)-3-methoxypiperidin-1-yl)-4-(1-(cyclopropylmethyl)-1H-1,2,4-triazol-5-yl)thiazole-5-carboxylic acid;
2-((3S,4R)-4-(3-bromo-4-chloro-5-methyl-1H-pyrrole-2-carboxamido)-3-methoxypiperidin-1-yl)-4-(1-(2-methoxyethyl)-1H-1,2,4-triazol-5-yl)thiazole-5-carboxylic acid;
2-((3S,4R)-4-(3-bromo-4-chloro-5-methyl-1H-pyrrole-2-carboxamido)-3-fluoropiperidin-1-yl)-4-(1-(2-methoxyethyl)-1H-1,2,4-triazol-5-yl)thiazole-5-carboxylic acid;
2-((3S,4R)-4-(4-bromo-3-chloro-5-methyl-1H-pyrrole-2-carboxamido)-3-methoxypiperidin-1-yl)-4-(1-(2-methoxyethyl)-1H-1,2,4-triazol-5-yl)thiazole-5-carboxylic acid; and
2-((3S,4R)-4-(3,5-dichloro-4-methyl-1H-pyrrole-2-carboxamido)-3-methoxypiperidin-1-yl)-4-(1-(2-methoxyethyl)-1H-1,2,4-triazol-5-yl)thiazole-5-carboxylic acid,
   or a pharmaceutically acceptable salt thereof.

In one embodiment of the invention are provided compounds of formula (I), in an alternative embodiment are provided pharmaceutically-acceptable salts of compounds of formula (I)

In a further aspect the present invention provides a process for preparing a compound of formula (I) or a pharmaceutically-acceptable salt thereof.

Thus, the present invention also provides that the compounds of the formula (I) and pharmaceutically-acceptable salts thereof, can be prepared by a process as follows (wherein the variables are as defined above unless otherwise stated):
(a) reacting a compound of formula (II):

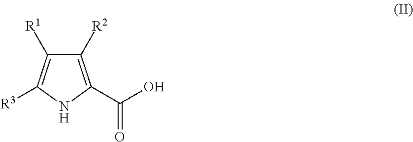

or an activated acid derivative thereof; with a compound of formula (III):

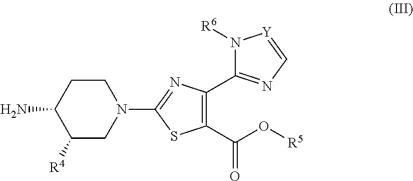

or
(b) reacting a compound of formula (IV)

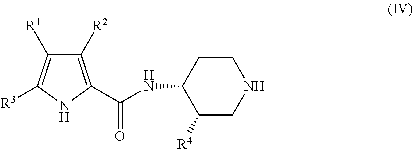

with a compound of formula (V):

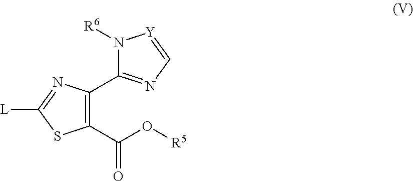

wherein L is a displaceable group; or
(c) for compounds of formula (I) wherein R⁵ is C₁₋₄alkyl; reacting a compound of formula (I) which is a compound of formula (VI)

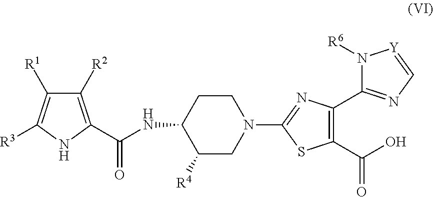

is with a compound of formula (VII):

R^{4a}—OH (VII)

wherein R^{4a} is C₁₋₄alkyl;
or
(d) for compounds of formula (I) wherein R⁵ is hydrogen; deprotecting a compound of formula (VIII)

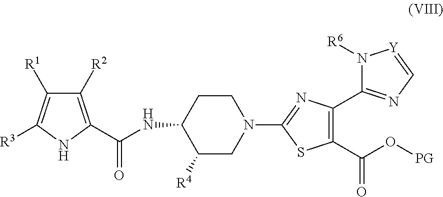

wherein PG is a carboxylic acid protecting group;
and thereafter if necessary:
i) converting a compound of the formula (I) into another compound of the formula (I);
ii) removing any protecting groups;
iii) forming a pharmaceutically acceptable salt; and/or
iv) chirally purifying the compound of formula (I).

L is a displaceable group. Suitable values for L include halo, for example chloro and bromo, pentafluorophenoxy and 2,5-oxopyrrolidin-1-yloxy.

PG is a carboxylic acid protecting group. Suitable values for PG are defined herein below.

Specific reaction conditions for the above reaction are as follows.

(a) Compounds of formula (II) or and (III) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (III) may be prepared according to Scheme 1:

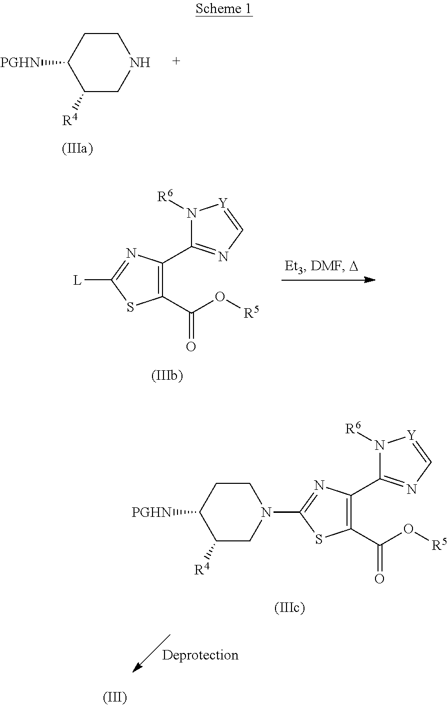

wherein PG is a nitrogen protecting group such as those defined herein below; and L is a displaceable group such as those defined herein above.

Compounds of formula (II) are commercially available compounds, or they are known in the literature, or they are prepared by standard processes known in the art.

(b) Compounds of formula (IV) and (V) in a suitable solvent such as dimethylformamide or N-methylpyrrolindine and optionally in the presence of a base such as triethylamine or diisopropylamine are heated together at a temperature range between 50 to 100° C.

Compounds of formula (IV) may be prepared according to Scheme 2:

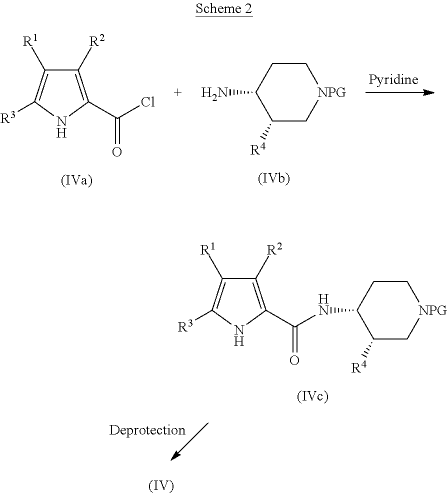

wherein PG is a nitrogen protecting group such as those defined herein below.

Compounds of formula (V) may be prepared according to Scheme 3:

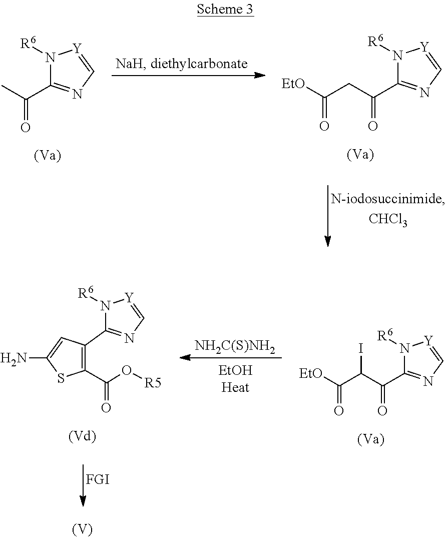

wherein FGI is functional group interconversion of the NH₂ group to the required “L”.

(c) Compounds of formula (VI) and (VII) in a suitable solvent such as methanol, ethanol, or tetrahydrofuran in the presence of a base such as sodium hydroxide, lithium hydroxide, or barium hydroxide are reacted at a temperature range of 25 to 100° C.

Compounds of formula (VI) may be prepared by a suitable modification of the reactions described herein to make a compound of formula (I) wherein R⁴ is hydrogen.

Compounds of formula (VII) are commercially available compounds, or they are known in the literature, or they are prepared by standard processes known in the art.

(d) Suitable deprotection conditions are described hereinbelow.

Compounds of formula (VIII) may be prepared by a suitable modification of the reactions described herein to make a compound of formula (I).

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I)) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

Optically active forms of a compound of the invention may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the formula (I) or a pharmaceutically-acceptable salt thereof for use in a method of treatment of the human or animal body by therapy.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention the compounds of the invention inhibit topoisomerase IV and are therefore of interest for their antibacterial effects. In one aspect of the invention the compounds of the invention inhibit both DNA gyrase and topoisomerase IV and are therefore of interest for their antibacterial effects.

It is expected that the compounds of the present invention will be useful in treating bacterial infections. In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficili. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptococcus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis.

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens.

According to a further feature of the present invention the “infection” or “bacterial infection” refers to an infection caused by a mycobacterium and in particular any one of Mycobacterium tuberculosis (Mtu), M. avium intracellulare (Mai) and M. ulcerans (Mul)

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a zo bacterial infection in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being, in need of such treatment which comprises administering to said animal an effective amount of a compound of formula (I) or a pharmaceutically acceptable salt thereof as defined hereinbefore.

A further feature of the present invention is a compound of formula (I) and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for the production of an anti-bacterial effect in a warm-blooded animal such as a human being.

According to a further aspect of the invention there is provided the use of a compound of formula (I), or a pharmaceutically acceptable salt thereof in the manufacture of a medicament for the inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof in the manufacture of a medicament for the treatment of a bacterial infection in a warm-blooded animal such as a human being.

According to a further feature of the invention there is provided a method of treating a bacterial infection selected from pulmonary tuberculosis, extra-pulmonary tuberculosis, avium infections, Buruli ulcer in a warm-blooded animal, such as a human being, in need of such treatment which comprises administering to said animal an effective amount of a compound of formula (I) or a pharmaceutically acceptable salt thereof as defined hereinbefore.

Thus according to a further aspect of the invention there is provided the use of a compound of formula (I), or a pharmaceutically acceptable salt thereof in the manufacture of a medicament for the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

According to a further aspect of the invention there is provided a compound of formula (I), or a pharmaceutically acceptable salt thereof for use in the production of an anti-bacterial effect in a warm-blooded animal such as a human being.

According to a further aspect of the invention there is provided a compound of formula (I), or a pharmaceutically acceptable salt thereof for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof for use in the treatment of a bacterial infection in a warm-blooded animal such as a human being.

Thus according to a further aspect of the invention there is provided a compound of formula (I), or a pharmaceutically acceptable salt thereof for use in the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

In order to use a compound of the formula (I) or a pharmaceutically-acceptable salt thereof, for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or a pharmaceutically-acceptable salt thereof, and a pharmaceutically-acceptable diluent or carrier.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in producing an anti-bacterial effect in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in inhibition of bacterial DNA gyrase and/or topoisomerase IV in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a bacterial infection in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. In one aspect of the invention a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) or (Ia) and their pharmaceutically acceptable salts are also useful as pharmacological tools in the development and standardisation of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Combinations

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) one or more antibacterial agents useful in the treatment of Mycobacterium tuberculosis such as one or more of rifampicin, isoniazid, pyrizinamide, ethambutol, quinolones e.g. moxifloxacin or gatifloxacin, streptomycin.
v) efflux pump inhibitors.

Therefore, in a further aspect of the invention there is provided a compound of the formula (I), or a pharmaceutically acceptable salt thereof and a chemotherapeutic agent selected from:

i) one or more additional antibacterial agents; and/or
ii) one or more anti-infective agents; and/or
iii) biological protein therapeutics for example antibodies, cytokines, bactericidal/permeability-increasing protein (BPI) products;
iv) one or more antibacterial agents useful in the treatment of pulmonary tuberculosis, extra-pulmonary tuberculosis, avium infections, buruli ulcers and/or
v) one or more efflux pump inhibitors.

### EXAMPLES

The invention is now illustrated but not limited by the following Examples in which unless otherwise stated:—

(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR zo and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad;
(vi) fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ES); values for m/z are given; generally, only ions which indicate the parent mass are reported;
(vii) each intermediate was generally purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:
DMF is N,N-dimethylformamide;
SM is starting material;
DMSO is dimethylsulfoxide;
CDCl₃ is deuterated chloroform;
MS is mass spectroscopy;
EtOAc is ethyl acetate;
THF is tetrahydrofuran;
MeOH is methanol;
TFA is trifluoroacetic acid;
EtOH is ethanol;
DCM is dichloromethane;
HATU is N-[(dimethylamino)-1H,2,3-triazolo[4,5-b]pyridin-1-ylmethylene]-N-methylmethanaminium hexafluorophosphate N-oxide;
DIEA is diisopropyl ethyl amine; and
   (viii) temperatures are quoted as ° C.

### Example 1

### 2-((3S,4R)-4-{[3-Bromo-4-chloro-5-methyl-1H-pyrrole-2-carbonyl]amino}-3-methoxypiperidin-1-yl)-4-(1-methyl-1H-1,2,4-triazol-5-yl)-1,3-thiazole-5-carboxylic acid

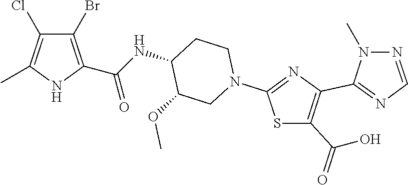

To a suspension of ethyl 2-((3S,4R)-4-(3-bromo-4-chloro-5-methyl-1H-pyrrole-2-carboxamido)-3-methoxypiperidin-1-yl)-4-(1-methyl-1H-1,2,4-triazol-5-yl)thiazole-5-carboxylate (Intermediate 1, 520 mg, 0.89 mmol) in THF (16.00 mL) and EtOH (4 mL) was added LiOH (212 mg, 8.86 mmol) in water (2 mL) and heated to 60° C. for overnight. The progress of the reaction was monitored through LCMS and the LCMS profile showed the completion of reaction after heating to 60° C. for overnight. Reaction mixture was concentrated under vacuum and the residue was dissolved in water and acidified with 6N HCl (pH 4). The resulting precipitate was filtered, washed with water, and dried (450 mg, 91%).

MS (ES) (M+H)⁺: 559 for C₁₉H₂₁BrClN₇O₄S

NMR: 1.83 (m, 2H), 2.21 (s, 3H), 3.35-3.45 (m, 5H), 3.63 (m, 1H), 4.05 (m, 1H), 4.13 (s, 3H), 4.37 (m, 2H), 7.33 (d, 1H), 8.23 (s, 1H), 12.35 (s, 1H), 15.5, (bs, 1H).

### Examples 2-22

The following Examples were prepared by the procedure described in Example 1 from the starting materials (SM) indicated.

| Ex | Compound | Data | SM |
|---|---|---|---|
| 2 | 2-((3S,4R)-4-{[4-Bromo-3-chloro-5-methyl-1H-pyrrole-2- carbonyl]amino}-3-methoxypiperidin-1-yl)-4-(1- methyl-1H-1,2,4-triazol-5-yl)-1,3- thiazole-5-carboxylic acid 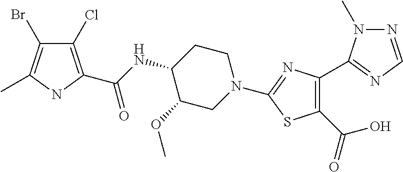 | MS(ES) (M + H)⁺: 559 for C₁₉H₂₁BrClN₇O₄S NMR: 1.81 (m, 2H), 2.20 (s, 3H), 3.31-3.40 (m, 5H), 3.65 (m, 1H), 4.0 (m, 1H), 4.10 (s, 3H), 4.38 (m, 2H), 7.20 (d, 1H), 8.24 (s, 1H), 12.20 (s, 1H). 15.45, (bs, 1H). | Intermediate 2 |
| 3 | 2-((3S,4R)-4-{[(3-bromo-4-chloro-5-methyl-1H-pyrrol-2-yl) carbonyl]amino}-3-methoxypiperidin-1-yl)- 4-(1-methyl-1H-imidazol-2-yl)-1,3- thiazole-5-carboxylic acid 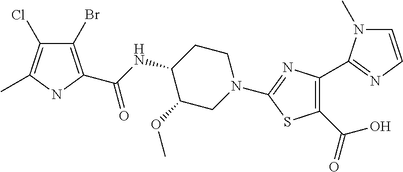 | MS (ES) (M + H)⁺: 558 for C₂₀H₂₂BrClN₆O₄S NMR: 1.83 (m, 2H), 2.21 (s, 3H), 3.40-3.45 (m, 5H), 3.61 (m, 1H), 4.02 (m, 1H), 4.15 (s, 3H), 4.35 (m, 2H), 7.27 (d, 1H), 7.32 (s, 1H), 7.51 (s, 1H), 12.20 (s, 1H). | Intermediate 3 |
| 4 | 2-((3S,4R)-4-{[(3,5-Dichloro-4-methyl-1H-pyrrol-2-yl) carbonyl]amino}-3-methoxypiperidin-1-yl)-4-(1- methyl-1H-1,2,4-triazol-5-yl)-1,3-thiazole-5- carboxylic acid 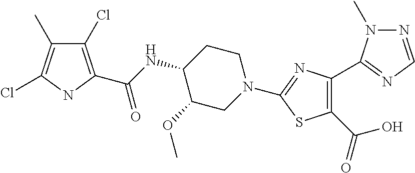 | MS (ES) (M + H)⁺: 514 for C₁₉H₂₁Cl₂N₇O₄S NMR: 1.81 (m, 2H) 1.98 (s, 3H), 3.33-3.42 (m, 5H), 3.60 (m, 1H), 3.97 (m, 1H), 4.12 (s, 3H), 4.34 (m, 2H), 7.21 (d, 1H), 8.25 (s, 1H), 12.60 (s, 1H). 15.45, (s, 1H). | Intermediate 4 |
| 5 | 2-((3S,4R)-4-{[(3-Bromo-4-chloro-5-methyl-1H-pyrrol-2-yl) carbonyl]amino}-3-fluoropiperidin-1-yl)-4- (1-methyl-1H-1,2,4-triazol-5-yl)-1,3-thiazole- 5-carboxylic acid 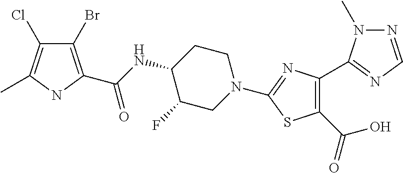 | MS (ES) (M + H)⁺: 547 for C₁₈H₁₈Cl₂FN₇O₃S NMR: 1.91 (m, 2H), 2.22 (s, 3H), 3.45 (m, 1H), 3.63 (dd, 1H), 4.11 (m, 4H), 4.32-4.47 (m, 2H), 5.00 (d, 1H), 7.68 (d, 1H), 8.22 (s, 1H), 12.55 (s, 1H). | Intermediate 5 |
| 6 | 2-((3S,4R)-4-{[(4-Bromo-3-chloro-5-methyl-1H-pyrrol-2-yl) carbonyl]amino}-3-fluoropiperidin-1-yl)-4-(1-methyl- 1H-1,2,4-triazol-5-yl)-1,3-thiazole-5-carboxylic acid 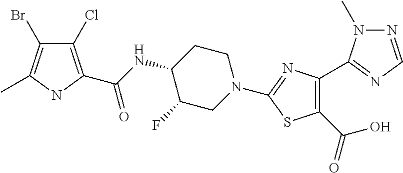 | MS (ES) (M + H)⁺: 547 for C₁₈H₁₈Cl₂FN₇O₃S NMR: 1.90 (m, 2H), 2.20 (s, 3H), 3. 3.40 (m, 1H), 3.70 (dd, 1H), 4.10 (m, 4H), 4.32-4.47 (m, 2H), 5.00 (d, 1H), 7.27 (d, 1H), 8.22 (s, 1H), 12.17 (s, 1H), 15.40 (bs, 1H). | Intermediate 6 |
| 7 | 2-((3S,4R)-4-{[(3-Bromo-4-chloro-5-methyl-1H-pyrrol-2-yl) carbonyl]amino}-3-methoxypiperidin-1-yl)- 4-[1-(2-methoxyethyl)-1H-imidazol-2-yl]-1,3- thiazole-5-carboxylic acid 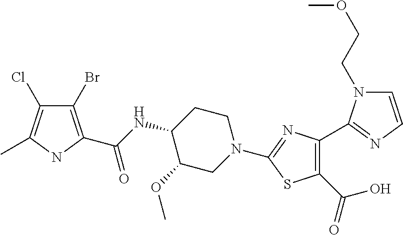 | MS (ES) (M + H)⁺: 602 for C₂₂H₂₆BrClN₆O₅S NMR: 1.85 (m, 2H), 2.22 (s, 3H), 3.22 (s, 3H), 3.40-3.50 (m, 4H), 3.61 (m, 1H), 3.74 (t, 2H), 4.03 (m, 1H), 4.31 (m, 2H), 4.82 (t, 2H), 7.30 (d, 1H), 7.40 (s, 1H), 7.67 (s, 1H), 12.22 (s, 1H). | Intermediate 7 |
| 8 | 2-{(3S,4R)-4-[(3-Bromo-4-cyano-5-methyl-1H-pyrrole- 2-carbonyl)-amino]-3-methoxy-piperidin-1-yl}-4-(2- methyl-2H-[1,2,4]triazol-3-yl)-thiazole-5-carboxylic acid 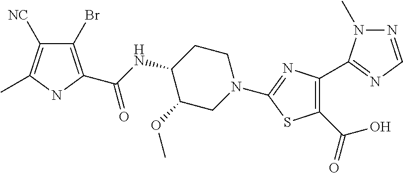 | MS (ES) (M + 2)⁺: 551.2 for C₂₀H₂₁BrN₈O₄S 1H NMR (300 MHz, DMSO- d₆). 81 (d, J = 3.20 Hz, 2H) 2.35 (s, 3H) 3.40 (s, 4H) 3.46 (br. s., 1H) 3.60 (br. s., 1H) 4.02 (br. s., 1H) 4.10 (s, 3H) 4.30 (d, J = 8.10 Hz, 2H) 7.37 (d, J = 7.91 Hz, 1H) 8.24 (s, 1H) 12.68 (s, 1H) 15.44 (br. s., 1H) | Intermediate 8 |
| 9 | 2-{(3S,4R)-4-[(4-Bromo-3-cyano-5-methyl-1H- pyrrole-2-carbonyl)-amino]-3-methoxy-piperidin-1-yl}- 4-(2-methyl-2H-[1,2,4]triazol-3-yl)-thiazole-5- carboxylic acid 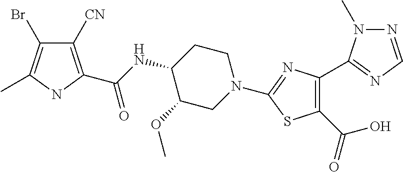 | MS (ES) (M + 2)⁺: 551.2 for C₂₀H₂₁BrN₈O₄S 1H NMR (300 MHz, DMSO- d₆) 1.86 (dd, J = 12.72 Hz, 1H) 1.77 (d, J = 3.77 Hz, 1H) 2.21 (s, 3H) 3.31 (s, 3H) 3.45 (br. s., 2H) 3.60 (br. s., 1H) 3.99 (br.s., 1H) 4.09 (s, 3H) 4.29 (br. s., 2H) 7.75 (d, J = 7.91 Hz, 1H) 8.25 (s, 1H) 12.72 (br. s., 1H) 15.45 (br.s., 1H) | Intermediate 9 |
| 10 | 2-((3S,4R)-4-(3-bromo)-4-chloro-5-methyl-1H-pyrrole-2- carboxamido)-3-ethoxypiperidin-1-yl)-4- (1-methyl-1H-1,2,4-triazol-5-yl)thiazole- 5-carboxylic acid 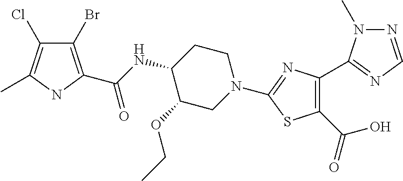 | MS (ES) (M + 2)⁺: 574.1 for C20H23BrClN7O4S 1H NMR (300 MHz, DMSO- d₆) 1.09 (t, 3H) 1.74-1.86 (m, 2H) 2.21 (s, 3H) 3.32- 3.55 (m, 3H) 3.63-3.75 (m, 2H) 4.10-4.00 (m, 1H) 4.10 (s, 3 H) 4.35-4.25 (m, 2H) 7.23 (d, J = 8.48 Hz, 1H) 8.25 (s, 1H) 12.20 (s, 1H) 15.45 (br. s., 1H) | Intermediate 10 |
| 11 | 2-((3S,4R)-4-(4-bromo-3-chloro-5-methyl-1H-pyrrole-2- carboxamido)-3-ethoxypiperidin-1-yl)-4-(1-methyl- 1H-1,2,4-triazol-5-yl) thiazole-5-carboxylic acid 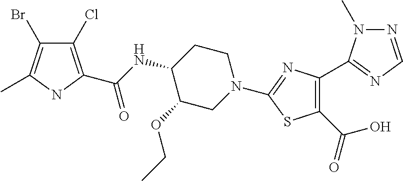 | MS (ES) (M + 2)⁺: 574.1 for C20H23BrClN7O4S 11H NMR (300 MHz, DMSO- d₆) 1.08 (t, J = 6.88 Hz, 3H) 1.81 (d, J = 3.39 Hz, 2H) 2.19 (s, 3H) 3.47 (dd, J = 9.61, 7.16 Hz, 2H) 3.42 (br. s., 2H) 3.69 (d, J = 9.42 Hz, 2H) 3.70 (br. s., 2H) 4.10 (s, 3H) 4.29 (d, J = 7.16 Hz, 2H) 7.14 (d, J = 8.48 Hz, 1H) 8.25 (s, 1H) 12.20 (s, 1H) 15.46 (br. s., 1H). | Intermediate 11 |
| 12 | 2-((3S,4R)-4-(3-bromo-4-chloro-5-methyl-1H-pyrrole-2- carboxamido)-3-(cyclopropylmethoxy) piperidin-1- yl)-4-(1-methyl-1H-1,2,4-triazol-5-yl) thiazole-5- carboxylic acid 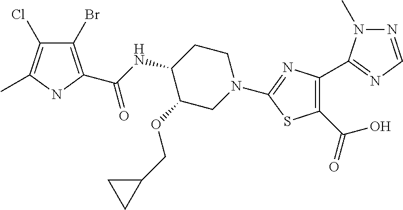 | M MS (ES) (M + H)⁺: 599 for C22H25BrClN7O4S NMR: 0.1 (m, 2H), 0.4 (m, 2H), 0.9 (m, 1H), 1.8 (m, 2H), 2.2 (s, 3H), 3.2-3.4 (m, 4H), 3.7 (m, 1H), 4.1 (s, 3H), 4.2- 4.4 (m, 1H), 7.2 (d, 1H), 8.2 (s, 1H), 12.2 (s, 1H). | Intermediate 12 |
| 13 | 2-((3S,4R)-4-(3-bromo-4-chloro-5-methyl-1H-pyrrole-2- carboxamido)-3-methoxypiperidin-1-yl)-4- (1-ethyl-1H-1,2,4-triazol-5-yl) thiazole-5- carboxylic acid 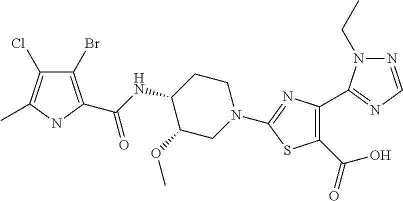 | MS (ES) (M + H)⁺: 573.7 for C20H23BrClN7O4S ¹H NMR (300 MHz, DMSO- d₆) 1.40 (t, 3H) 1.85-1.72 (m, 2H) 2.20 (s, 3H) 3.40 (s, 3H) 3.50-3.40 (m, 2H) 3.60- 3.58 (m, 1H) 4.10-3.92 (m, 1H) 4.40-4.25 (m, 2H) 4.60- 4.50 (q, 2H) 7.27 (d, 1H) 8.27 (s, 1H) 12.20 (s, 1H) 15.51 (bs, 1H) | Intermediate 13 |
| 14 | 2-((3S,4R)-4-(4-bromo-3-chloro-5-methyl-1H-pyrrole-2- carboxamido)-3-methoxypiperidin-1-yl)-4-(1-ethyl-1H- 1,2,4-triazol-5-yl) thiazole-5-carboxylic acid 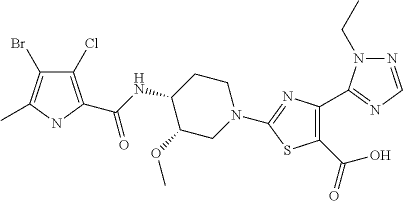 | MS (ES) (M + H)⁺: 573 for C20H23BrClN7O4S NMR: 1.3-1.5 (m, 3H), 1.6-1.7 (m, 2H), 2.2 (s, 3H), 3.3 (s, 3H), 3.6 (m, 1H), 4.9 (m, 1H), 4.2-4.4 (m, 2H), 4.5- 4.6 (m, 2H), 7.2 (d, 1H), 8.2 (s, 1H), 12.2 (s, 1H), 15.5 (s, 1H). | Intermediate 14 |
| 15 | 2-{(3S,4R)-4-[(3,5-Dichloro-4-methyl-1H-pyrrole-2- carbonyl)-amino]-3-methoxy-piperidin-1-yl}-4-(2- ethyl-2H-[1,2,4]triazol-3-yl)-thiazole-5-carboxylic acid 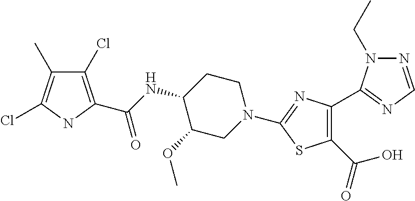 | MS (ES) (M + H)⁺: 529.8 for C₂₀H₂₃Cl₂N₇O₄S 1H NMR (300 MHz, DMSO- d₆) 1.39 (t, J = 7.16 Hz, 3H) 1.80 (d, J = 3.77 Hz, 2H) 1.93 (s, 3H) 3.44 (d, J = 13.56 Hz, 1H) 3.38 (s, 4H) 3.59 (br. s., 1H) 3.96 (d, J = 15.64 Hz, 1H) 4.28 (d, J = 9.80 Hz, 2H) 4.53 (dd, J = 7.35, 2.45 Hz, 2H) 7.21 (d, J = 8.29 Hz, 1H) 8.27 (s, 1H) 12.59 (s, 1H) 15.49 (br. s., 1H) | Intermediate 15 |
| 16 | 2-((3S,4R)-4-(3-bromo-4-chloro-5-methyl-1H-pyrrole-2- carboxamido)-3-methoxypiperidin-1-yl)-4-(1- (cyclopropylmethyl)-1H-1,2,4-triazole-5-yl) thiazole-5- carboxylic acid 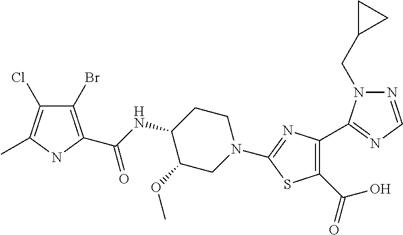 | MS (ES) (M + H)⁺: 598/600 for C22H25BrClN7O4S 1HNMR: 0.35(m, 2H), 0.5 (m, 2H), 1.3 (2h, m), 1.8 (2H, m), 2.25 (3H, s), 3.4 (5h, m), 3.6 (1H, s), 3.95 (1H, b), 4.35 (4H, m), 7.3 (1H, d), 8.2 (1H, s), 12.2 (1H, b) | Intermediate 16 |
| 17 | 2-((3S,4R)-4-(3-bromo-4-chloro-5-methyl-1H-pyrrole-2- carboxamido)-3-fluoropiperidin-1-yl)-4-(1- (cyclopropylmethyl)-1H-1,2,4-triazol-5-yl) thiazole-5- carboxylic acid 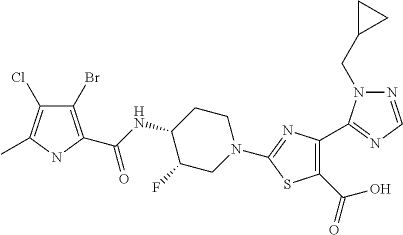 | MS (ES) (M + H)⁺: 598/600 for C2H22BrClFN7O3S HNMR: 0.35 (2H, m), 0.5 (2H, m), 1.3 (1H, m), 1.9 (2H, m), 2.2 (3H, s), 3.65 (3H, m), 4.05 (1H, d), 4.4 (4H, m), 5 (1H, d), 7.35 (1H, d), 8.3 (1H, s), 12.15 (1H, bs) | Intermediate 17 |
| 18 | 2-((3S,4R)-4-(4-bromo-3-chloro-5-methyl-1H-pyrrole-2- carboxamido)-3-methoxypiperidin-1-yl)-4-(1- (cyclopropylmethyl)-1H-1,2,4-triazol-5-yl) thiazole-5- carboxylic acid 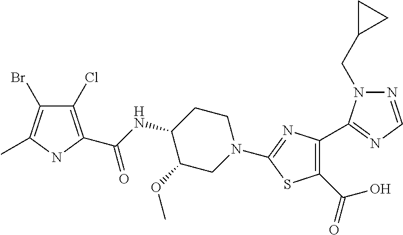 | MS (ES) (M + H)⁺: 598/600 for C22H25BrClN7O4S 1H NMR (300 MHz, DMSO- d₆) 0.3 (m, 2H) 0.51 (m, 2H) 1.34 (m, 1H) 1.75 (m, 2H) 2.22 (s, 3H) 3.35 (s, 3H) 3.45 (m, 2H) 3.6 (m, 1H) 4.01 (m, 1H) 4.21-4.40 (m, 4H) 7.2 (d, 1H) 8.35 (s, 1H) 12.2 (bs 1H) | Intermediate 18 |
| 19 | 2-((3S,4R)-4-(3-bromo-4-chloro-5-methyl-1H-pyrrole-2- carboxamido)-3-methoxypiperidin-1-yl)-4- (1-(2-methoxyethyl)-1H-1,2,4-triazol-5-yl) thiazole-5- carboxylic acid 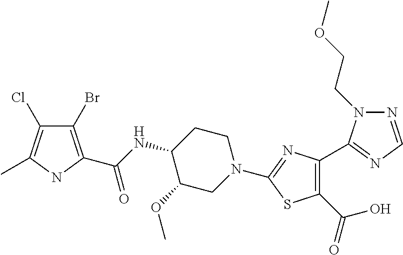 | MS (ES) (M + H)⁺: 603.7 for C21H25BrClN7O5S ¹H NMR (300 MHz, DMSO- d₆) 1.70-1.90 (m, 2H) 2.20 (s, 3H) 3.17 (s, 3H) 3.40 (s, 3H) 3.50-3.40 (m, 2H) 3.60- 3.55 (m, 1H) 3.75 (t, 2H) 4.10-3.92 (m, 1H) 4.40- 4.20 (m, 2H) 4.72 (t, 2H) 7.27 (d, 1H) 8.27 (s, 1H) 12.20 (s, 1H) 15.61 (bs, 1H) | Intermediate 19 |
| 20 | 2-((3S,4R)-4-(3-bromo-4-chloro-5-methyl-1H-pyrrole-2- carboxamido)-3-fluoropiperidin-1-yl)-4-(1-(2- methoxyethyl)-1H-1,2,4-triazol-5-yl) thiazole-5- carboxylic acid 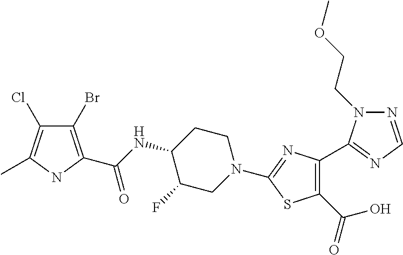 | MS (ES) (M + H)⁺: 592 for C20H222BrClFN7O4S 1HNMR: 1.9 (2H, m), 2.25 (3H, s), 3.15 (3H, s), 3.5 (4H, m), 4.0 (1H, dd), 4.45 (2H, m), 4.6 (2H, m), 5.0 (1H, d), 7.35 (1H, d), 8.3 (1H, s), 12.15 (1H, bs) | Intermediate 20 |
| 21 | 2-((3S,4R)-4-(4-bromo-3-chloro-5-methyl-1H-pyrrole-2- carboxamido)-3-methoxypiperidin-1-yl)-4-(1-(2-methoxyethyl)- 1H-1,2,4-triazol-5-yl) thiazole-5-carboxylic acid 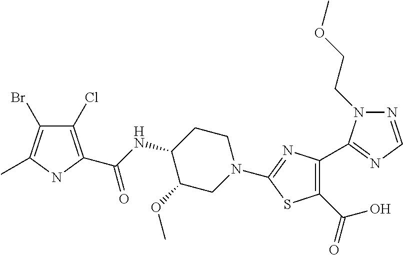 | MS (ES) (M + H)⁺: 604.22 for C₂₁H₂₅BrClN₇O₅S. 1H NMR (300 MHz, DMSO- d₆) 1.80 (d, J = 4.33 Hz, 2H) 2.19 (s, 3H) 3.18 (s, 3H) 3.34- 3.49 (m, 5H) 3.60 (d, J = 1.32 Hz, 1H) 3.75 (t, J = 5.65 Hz, 2H) 3.96 (br. s., 1H) 4.31 (br. s., 2H) 4.72 (t, J = 5.46 Hz, 2H) 7.18 (d, J = 8.10 Hz, 1H) 8.30 (s, 1H) 12.20 (s, 1H) 15.56 (br. s., 1H) | Intermediate 21 |
| 22 | 2-((3S,4R)-4-(3,5-dichloro-4-methyl-1H-pyrrole-2-carboxamido)- 3-methoxypiperidin-1-yl)-4-(1-(2-methoxyethyl)-1H-1,2,4-triazol-5-yl) thiazole-5-carboxylic acid 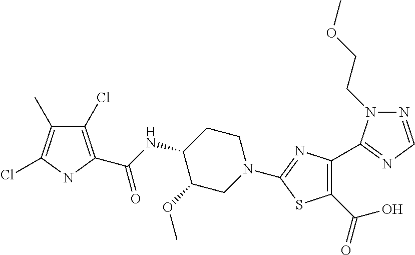 | MS (ES) (M + H)⁺: 560.32 for C₂₁H₂₅Cl₂N₇O₅S. 1H NMR (300 MHz, DMSO- d₆) 1.79 (br. s., 2H) 1.93 (s, 3H) 3.18 (s, 3H) 3.44 (d, J = 14.88 Hz, 1H) 3.38 (s, 4H) 3.59 (br. s., 1H) 3.75 (t, J = 5.56 Hz, 2H) 3.96 (br. s., 1H) 4.29 (d, J = 6.97 Hz, 2H) 4.72 (t, J = 5.65 Hz, 2H) 7.22 (d, J = 7.91 Hz, 1H) 8.30 (s, 1H) 12.61 (s, 1H) 15.55 (br. s., 1H) | Intermediate 22 |

### Intermediate 1

### Ethyl 2-((3S,4R)-4-{[(3-Brom-4-chloro-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylate

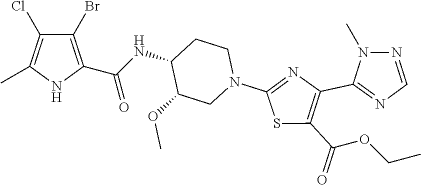

A suspension of 3-bromo-4-chloro-N-((3S,4R)-3-methoxypiperidin-4-yl)-5-methyl-1H-pyrrole-2-carboxamide (Intermediate 40, 600 mg, 1.71 mmol) in DMF (10 mL) was added DIPEA (0.897 mL, 5.13 mmol) and stirred. To this ethyl 2-chloro-4-(1-methyl-1H-1,2,4-triazol-5-yl)thiazole-5-carboxylate (Intermediate 23, 420 mg, 1.54 mmol) was added and heated to 60° C. for overnight. The progress of the reaction was monitored through LCMS and LCMS indicates conversion of starting material to product. Reaction mixture was concentrated under vacuum and the residue was dissolved in water and acidified with 6N HCl (pH 4). The precipitated solid was filtered, washed with water and dried under vacuum (520 mg, 51.8%).

MS (ES) (M+H)⁺: 588 for C₂₁H₂₅BrClN₇O₄S

NMR: 1.2 (t, 3H), 1.81 (m, 2, H), 2.20 (s, 3H), 3.40 (m, 4H), 3.57 (m, 1H), 3.75 (s, 3H), 4.0 (m, 1H), 4.11 (m, 2H), 4.31 (m, 2H), 7.15 (d, 1H), 8.0 (s, 1H), 12.20 (s, 1H).

### Intermediates 2-22

The following intermediate were prepared by the procedure described in Intermediate 1 from the starting materials (SM) indicated.

| Inter- | | | |
|---|---|---|---|
| mediate | Compound | Data | SM |
| 2 | Ethyl 2-((3S,4R)-4-{[(4-Brom- 3-chloro-5-methyl-1H-pyrrol-2- yl)carbonyl]amino}-3- methoxypiperidin-1-yl)-4-(1- methyl-1H-1,2,4-triazol-5-yl)- 1,3-thiazole-5-carboxylate 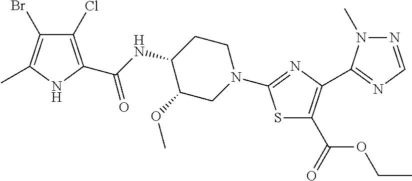 | MS (ES) (M + H)⁺: 588 for C₂₁H₂₅BrClN₇O₄S NMR: 1.21 (t, 3H), 1.81 (m, 2, H), 2.20 (s, 3H), 3.40 (m, 4H), 3.57 (m, 1H), 3.75 (s, 3H), 4.0 (m, 1H), 4.11 (m, 2H), 4.31 (m, 2H), 7.15 (d, 1H), 8.0 (s, 1H), 12.20 (s, 1H). | Intermediate 41 and intermediate 23 |
| 3 | Ethyl 2-((3S,4R)-4-{[(3- bromo-4-chloro-5-methyl-1H- pyrrol-2-yl)carbonyl]amino}- 3-methoxypiperidin-1-yl)-4-(1- methyl-1H-imidazol-4-yl)-1,3- thiazole-5-carboxylate 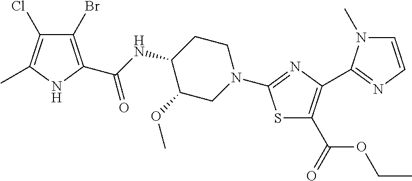 | MS (ES) (M + H)⁺: 587 for C₂₂H₂₆BrClN₆O₄S NMR: 1.22 (t, 3H), 1.75 (m, 2H), 2.23 (s, 3H), 3.34 (m, 4H), 3.48 (m, 1H), 3.71 (s, 3H), 4.03 (m, 1H), 4.13 (m, 2H), 4.34 (m, 2H), 7.15 (s, 1H), 7.61 (s, 1H), 8.04 (s, 1H), 12.20 (s, 1H). | Intermediate 40 and intermediate 24 |
| 4 | Ethyl 2-((3S,4R)-4-{[(3,5- dichloro--4-methyl-1H-pyrrol- 2-yl)carbonyl]amino}-3- methoxypiperidin-1-yl)-4-(1- methyl-1H-1,2,4-triazol-5-yl)- 1,3-thiazole-5-carboxylate 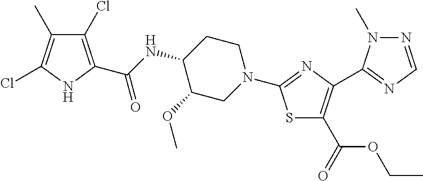 | MS (ES) (M + H)⁺: 543 for C₂₁H₂₅Cl₂N₇O₄S NMR: 1.19 (t, 3H), 1.81 (m, 2H), 1.98 (s, 3H), 3.33-3.42 (m, 5H), 3.60 (m, 1H), 3.97 (m, 1H), 4.12 (s, 3H), 4.16 (m, 2H), 4.34 (m, 2H), 7.21 (d, 1H), 8.25 (s, 1H), 12.60 (s, 1H). | Intermediate 42 and intermediate 23 |
| 5 | Ethyl 2-((3S,4R)-4-{[(3- Bromo-4-chloro-5-methyl-1H- pyrrol-2-yl)carbonyl]amino}- 3-fluoropiperidin-1-yl)-4-(1- methyl-1H-1,2,4-triazol-5-yl)- 1,3-thiazole-5-carboxylate 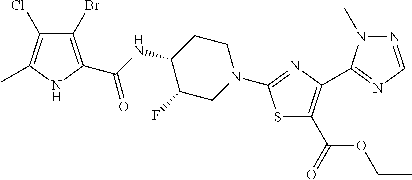 | MS (ES) (M + H)⁺: 576 lor C₂₀H₂₂BrClFN₇O₃S NMR: 1.20 (t, 3H), 1.91 (m, 2H), 2.22 (s, 3H), 3.45 (m, 1H), 3.63 (dd, 1H), 4.11 (m, 4H), 4.16 (m, 3H), 4.32-4.47 (m, 2H), 5.00 (d, 1H), 7.68 (d, 1H), 8.22 (s, 1H), 12.55 (s, 1H). | 3-bromo-4- chloro-N-((3S, 4R)-3- fluoropiperidin- 4-yl)-5-methyl- 1H-pyrrole-2- carboxamide (WO 2006087543 A1) and Intermediate 23 |
| 6 | Ethyl 2-((3S,4R)-4-{[(4- Bromo-3-chloro-5-methyl-1H- pyrrol-2-yl)carbonyl]amino}- 3-fluoropiperidin-1-yl)-4-(1- methyl-1H-1,2,4-triazol-5-yl)- 1,3-thiazole-5-carboxylate 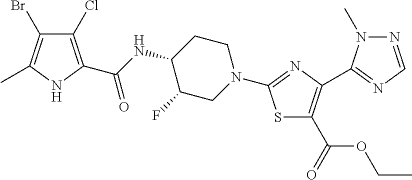 | MS (ES) (M + H)⁺: 576 for C₂₀H₂₂BrClFN₇O₃S NMR: 1.21 (t, 3H), 1.90 (m, 2H), 2.20 (s, 3H), 3. 3.40 (m, 1H), 3.70 (dd, 1H), 4.10 (m, 4H), 4.15 (m, 3H), 4.32-4.47 (m, 2H), 5.00 (d, 1H), 7.27 (d, 1H), 8.22 (s, 1H), 12.17 (s, 1H). | 4-bromo-3- chloro-N-((3S, 4R)-3- fluoropiperidin- 4-yl)-5-methyl- 1H-pyrrole-2- carboxamide (WO 2006087543 A1) and Intermediate 23 |
| 7 | Ethyl 2-((3S,4R)-4-{[(3- Bromo-4-chloro-5-methyl-1H- pyrrol-2-yl)carbonyl]amino}- 3-methoxypiperidin-1-yl)-4-[1- (2-methoxyethyl)-1H-imidazol- 2-yl]-1,3-thiazole-5-carboxylate 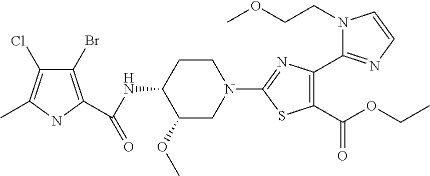 | MS (ES) (M + H)⁺: 631 For C₂₄H₃₀BrClN₆O₅S NMR: 1.10 (t, 3H), 1.82 (m, 2H), 2.21 (s, 3H), 3.21 (s, 3H), 3.34 (m, 4H), 3.60 (m, 3H), 4.0 (m, 1H), 4.10 (m, 4H), 4.33 (m, 2H), 7.05 (s, 1H), 7.33 (m, 2H), 12.20 (s, 1H). | Intermediate 40 and intermediate 25 |
| 8 | Ethyl 2-((3S,4R)-4-{[(3-Brom- 4-cyano-5-methyl-1H-pyrrol-2- yl)carbonyl]amino}-3- methoxypiperidin-1-yl)-4-(1- methyl-1H-1,2,4-triazol-5-yl)- 1,3-thiazole-5-carboxylate 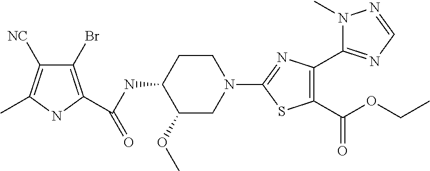 | MS (ES) (M + H)⁺: 558 for C₂₂H₂₅BrN₈O₄S | Intermediate 43 and intermediate 23 |
| 9 | Ethyl 2-((3S,4R)-4-{[(4-Brom- 3-cyano-5-methyl-1H-pyrrol-2- yl)carbonyl]amino}-3- methoxypiperidin-1-yl)-4-(1- methyl-1H-1,2,4-triazol-5-yl)- 1,3-thiazole-5-carboxylate 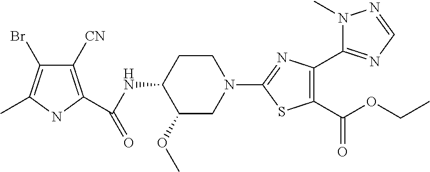 | MS (ES) (M + H)⁺: 558 for C₂₂H₂₅BrN₈O₄S | Intermediate 44 and intermediate 23 |
| 10 | Ethyl 2-((3S,4R)-4-{[(3- Bromo-4-chloro-5-methyl-1H- pyrrol-2-yl)carbonyl]amino}- 3-ethoxypiperidin-1-yl)-4-[1-(2- methoxyethyl)-1H-imidazol-2- yl]-1,3-thiazole-5-carboxylate 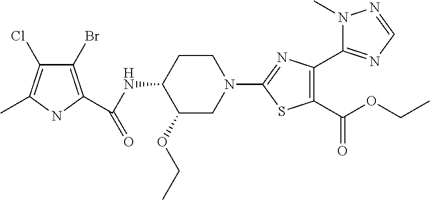 | MS (ES) (M + H)⁺: 601.7 for C22H27BrClN7O4S ¹H NMR (300 MHz, DMSO-d₆) 1.10 (t, 6H) 1.85-1.70 (m, 2 H) 2.20 (s, 3 H) 3.55-3.35 (m, 3H) 3.75-3.60 (m, 2H) 3.75 (s, 3H) 4.10-3.90 (m, 1H) 4.10 (q, 2H) 4.45- 4.20 (m, 2H) 7.25 (d, 1 H) 8.00 (s, 1 H) 12.20 (bs, 1 H) | Intermediate 44 and intermediate 23 |
| 11 | Ethyl 2-((3S,4R)-4-{[(4- Bromo-3-chloro-5-methyl-1H- pyrrol-2-yl)carbonyl]amino}- 3-ethoxypiperidin-1-yl)-4-[1-(2- methoxyethyl)-1H-imidazol-2- yl]-1,3-thiazole-5-carboxylate 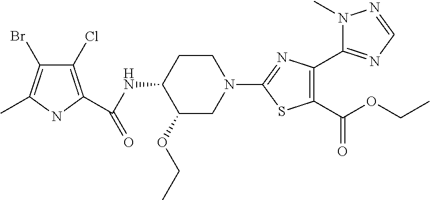 | MS (ES) (M + H)⁺: 601 for C22H27BrClN7O4S ¹H NMR (300 MHz, DMSO-d₆) 1.11 (t, 6H) 1.85-1.70 (m, 2 H) 2.20 (s, 3 H) 3.55-3.35 (m, 3H) 3.75-3.63 (m, 2H) 3.75 (s, 3H) 4.10-3.90 (m, 1H) 4.10 (q, 2H) 4.45- 4.20 (m, 2 H) 7.24 (d, 1 H) 8.00 (s, 1 H) 12.3 (bs, 1 H) | Intermediate 45 and intermediate 23 |
| 12 | Ethyl 2-((3S,4R)-4-(3-bromo- 4-chloro-5-methyl-1H-pyrrole- 2-carboxamido)-3- (cyclopropylmethoxy) piperidin-1-yl)-4-(1-methyl-1H- 1,2,4-triazol-5-yl)thiazole-5- carboxylate 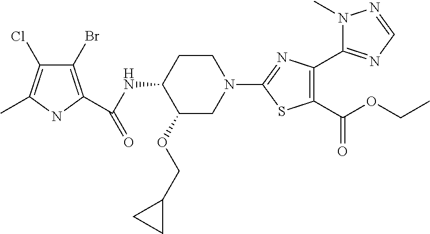 | MS (ES) (M + H)⁺: 627 for C24H29BrClN7O4S. | Intermediate 46 and intermediate 23 |
| 13 | Ethyl 2-((3S,4R)-4-(3-bromo- 4-chloro-5-methyl-1H-pyrrole- 2-carboxamido)-3- methoxypiperidin-1-yl)-4-(1- ethyl-1H-1,2,4-triazol-5-yl) thiazole-5-carboxylate 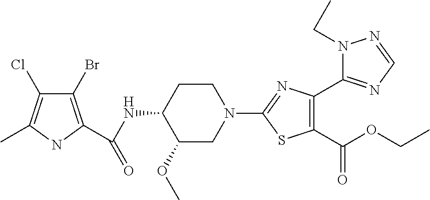 | MS (ES) (M + H)⁺: 602.2 for C22H27BrClN7O4S ¹H NMR (300 MHz, DMSO-d₆) 1.05 (t, 3H) 1.30 (t, 3H) 1.82-1.70 (m, 2 H) 2.20 (s, 3 H) 3.35 (s, 3H) 3.45-3.35 (m, 2H) 3.60-3.55 (m, 1H) 4.10-3.92 (m, 5H) 4.45-4.20 (m, 2 H) 7.30 (d, 1 H) 8.03 (s, 1 H) 12.17 (bs, 1 H) | Intermediate 40 and intermediate 75 |
| 14 | Ethyl 2-((3S,4R)-4-(4-bromo- 3-chloro-5-methyl-1H-pyrrole- 2-carboxamido)-3- methoxypiperidin-1-yl)-4-(1- ethyl-1H-1,2,4-triazol-5-yl) thiazole-5-carboxylate 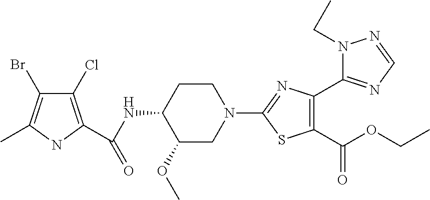 | MS(ES) (M + H)⁺: 602.2 for C22H27BrClN7O4S ¹H NMR (300 MHz, DMSO-d₆) 1.06 (t, 3H) 1.30 (t, 3H) 1.82-1.70 (m, 2 H) 2.20 (s, 3 H) 3.35 (s, 3H) 3.45-3.35 (m, 2H) 3.60-3.55 (m, 1H) 4.10-3.92 (m, 5H) 4.45-4.23 (m, 2 H) 7.30 (d, 1 H) 8.03 (s, 1 H) 12.18 (bs, 1 H) | Intermediate 41 and intermediate 75 |
| 15 | Ethyl 2-((3S,4R)-4-(3,5- dichloro-4-methyl-1H-pyrrole- 2-carboxamido)-3- methoxypiperidin-1-yl)-4-(1- ethyl-1H-1,2,4-triazol-5-yl) thiazole-5-carboxylate 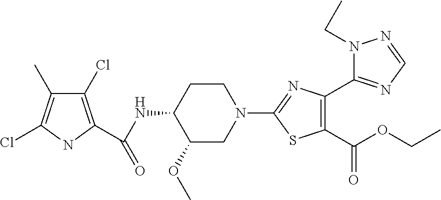 | MS (ES) (M + H)⁺: 556 for C₂₂H₂₇Cl₂N₇O₄S | Intermediate 42 and intermediate 75 |
| 16 | Ethyl 2-((3S,4R)-4-(3-bromo- 4-chloro-5-methyl-1H-pyrrole- 2-carboxamido)-3- methoxypiperidin-1-yl)-4-(1- (cyclopropylmethyl)-1H-1,2,4- triazol-5-yl)thiazole-5- carboxylate 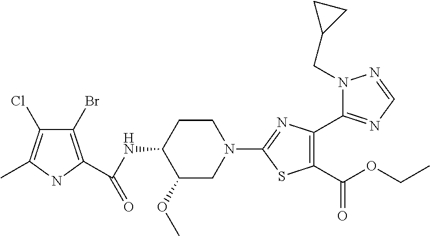 | MS (ES) (M + H)⁺: 627.7 for C24H29BrClN7O4S | Intermediate 40 and intermediate 80 |
| 17 | Ethyl 2-((3S,4R)-4-(3-bromo- 4-chloro-5-methyl-1H-pyrrole- 2-carboxamido)-3- fluoropiperidin-1-yl)-4-(1- (cyclopropylmethyl)-1H-1,2,4- triazol-5-yl)thiazole-5- carboxylate 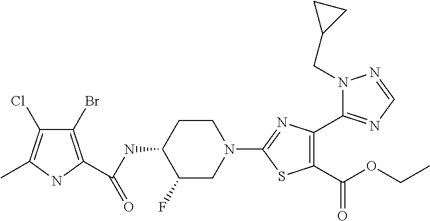 | MS (ES) (M + H)⁺: 615 for C23H26BrClFN7O3S | 3-bromo-4- chloro-N-((3S, 4R)-3- fluoropiperidin- 4-yl)-5-methyl- 1H-pyrrole-2- carboxamide (WO 2006087543 A1) and intermediate 80 |
| 18 | Ethyl 2-((3S,4R)-4-(4-bromo- 3-chloro-5-methyl-1H-pyrrole- 2-carboxamido)-3- methoxypiperidin-1-yl)-4-(1- (cyclopropylmethyl)-1H-1,2,4- triazol-5-yl)thiazole-5- carboxylate 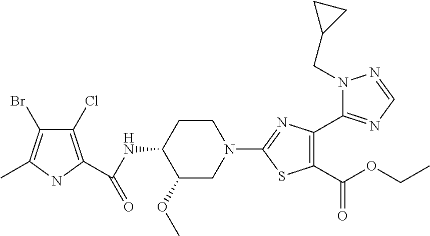 | MS (ES) (M + H)⁺: 627.7 for C24H29BrClN7O4S | Intermediate 41 and intermediate 80 |
| 19 | Ethyl 2-((3S,4R)-4-(3-bromo- 4-chloro-5-methyl-1H-pyrrole- 2-carboxamido)-3- methoxypiperidin-1-yl)-4-(1-(2- methoxyethyl)-1H-1,2,4- triazol-5-yl)thiazole-5- carboxylate 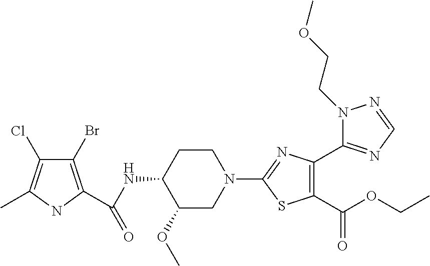 | MS (ES) (M + H)⁺: 631.7 for C23H29BrClN7O5S ¹H NMR (300 MHz, DMSO-d₆) 1.05 (t, 3H) 1.90-1.80 (m, 2 H) 2.18 (s, 3 H) 3.10 (s, 3H) 3.35 (s, 3H) 3.45-3.35 (m, 2H) 3.60-3.55 (m, 1H) 3.65 (t, 2H) 4.00-3.90 (m, 1 H) 4.10 (q, 2 H) 4.18 (t, 2 H) 4.40-4.25 (m, 2 H) 7.28 (d, 1 H) 8.02 (s, 1 H) 12.17 (bs, 1 H) | Intermediate 40 and intermediate 85 |
| 20 | Ethyl 2-((3S,4R)-4-(3-bromo- 4-chloro-5-methyl-1H-pyrrole- 2-carboxamido)-3- fluoropiperidin-1-yl)-4-(1-(2- methoxyethyl)-1H-1,2,4- triazol-5-yl)thiazole-5- carboxylate 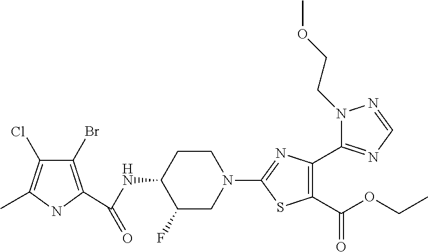 | MS (ES) (M + H)⁺: 619 for C₂₂H₂₆BrClFN₇O₄S | 3-bromo-4- chloro-N- ((3S,4R)-3- fluoropiperidin- 4-yl)-5-methyl- 1H-pyrrole-2- carboxamide (WO 2006087543 A1) and intermediate 85 |
| 21 | Ethyl 2-((3S,4R)-4-(4-bromo- 3-chloro-5-methyl-1H-pyrrole- 2-carboxamido)-3- methoxypiperidin-1-yl)-4-(1-(2- methoxyethyl)-1H-1,2,4- triazol-5-yl)thiazole-5- carboxylate 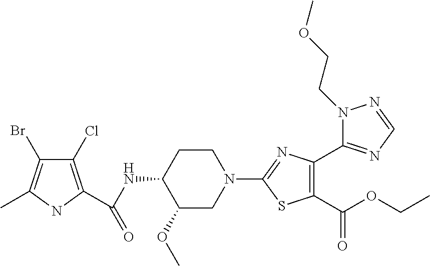 | MS (ES) (M + H)⁺: 631 For C₂₄H₃₀BrClN₆O₅S NMR: 1.11 (t, 3H), 1.83 (m, 2H), 2.21 (s, 3H), 3.21 (s, 3H), 3.34 (m, 4H), 3.60 (m, 3H), 4.01 (m, 1H), 4.10 (m, 4H), 4.33 (m, 2H), 7.05 (s, 1H), 7.33 (m, 2H), 12.23 (s, 1H). | Intermediate 41 and intermediate 85 |
| 22 | Ethyl 2-((3S,4R)-4-(3,5- dichloro-4-methyl-1H-pyrrole-2- carboxamido)-3- methoxypiperidin-1-yl)-4-(1-(2- methoxyethyl)-1H-1,2,4- triazol-5-yl)thiazole-5- carboxylate 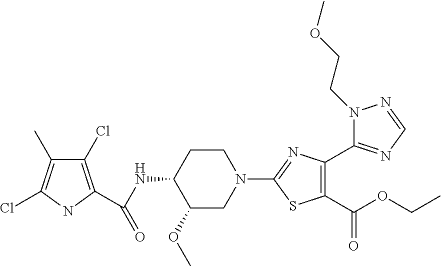 | MS (ES) (M + H)⁺: 586 for C₂₃H₂₉Cl₂N₇O₅S | Intermediate 42 and intermediate 85 |

### Intermediate 23

### Ethyl 2-chloro-4-(1-methyl-1H-1,2,4-triazol-5-yl)-1,3-thiazole-5-carboxylate

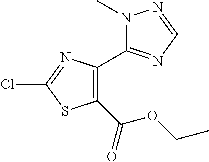

Copper (II) chloride (4.03 g, 30.00 mmol) and tert-butyl nitrite (3.57 mL, 30.00 mmol) were suspended in acetonitrile (40 mL) to give brownish green suspension. To this ethyl 2-amino-4-(1-methyl-1H-1,2,4-triazol-5-yl)thiazole-5-carboxylate (Intermediate 26, 3.80 g, 15 mmol) was added portion wise and resulting reaction mixture was stirred at 50° C. for 3 hrs. The progress of reaction was monitored by LCMS and LCMS profile showed completion of reaction after 3 hr stirring at 50° C. The reaction mixture was poured in to crushed ice and acidified with 6N HCl (pH 2.0). The resulting mixture was extracted with ethyl acetate (3×30 ml), dried over anhydrous sodium sulphate and evaporated afforded pure product as greenish yellowish oil which become solid upon cooling (3.9 gm)

MS (ES) (M+H)⁺: 273 for C₉H₉ClN₄O₂S

NMR: 1.20 (t, 3H), 3.82 (s, 3H), 4.21.21 (m, 2H), 8.10 (s, 1H).

### Intermediates 24-25

The following Intermediates were prepared by the procedure described in Intermediate 8 from the starting materials (SM) indicated.

| | Compound | Data | SM |
|---|---|---|---|
| 24 | Ethyl 2-chloro-4-(1-methyl-1H- imidazol-2-yl)-1,3-thiazole-5- carboxylate 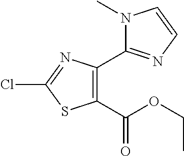 | MS (ES) (M + H)⁺: 272 for C₁₀H₁₀ClN₃O₂S NMR: 1.17 (t, 3H), 3.60 (s, 3H), 4.21 (m, 2H), 7.0 (s, 1H), 7.30 (s, 1H). | Intermediate 27 |
| 25 | Methyl 2-chloro-4-[1-(2- methoxyethyl)-1H-imidazol-2- yl]-1,3-thiazole-5-carboxylate 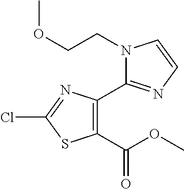 | MS (ES) (M + H)⁺: 302 for C₁₁H₁₂ClN₃O₃S NMR: 1.17 (t, 3H), 3.15 (s, 3H), 3.50 (m, 2H), 4.07 (m, 2H), 4.20 (m, 2H), 7.0 (s, 2), 7.35 (s, 1H). | Intermediate 28 |

### Intermediate 26

### Ethyl 2-Amino-4-(1-methyl-1H-1,2,4-triazol-5-yl)-1,3-thiazole-5-carboxylate

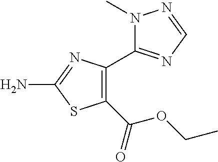

Sulfuryl dichloride (1.687 mL, 21.00 mmol) was added dropwise through the droping funnel over a period of 10 mints to a solution of ethyl 3-(1-methyl-1H-1,2,4-triazol-5-yl)-3-oxopropanoate (Intermediate 29, 3.94 g, 20 mmol) in 15 ml of DCM at 0° C. and the resulting the reaction mixture was stirred at room temperature for 1 hr. The reaction mixture was evaporated in vacou after 1 h stirring at room temperature. Thiourea (2.284 g, 30.00 mmol) and ethanol (30 mL) was added to the residue and refluxed for 6 hrs. The reaction was monitored by LCMS and LCMS profile showed completion of reaction after 6 hrs of reaction. The reaction mixture was cooled and concentrated in vacuo. Ice cooled water was added to the residue, sonicated well and neutralized with saturated sodium carbonate (20 ml). The precipitated solid was filtered, washed with water, ether and dried under high vacuum afforded the product as off white solid (4.8 gm).

MS (ES) (M+H)⁺: 254 for C₉H₁₁N₅O₂S

NMR: 1.11 (t, 3H), 3.61 (s, 3H), 4.10 (m, 2H), 7.97 (s, 1H), 8.07 (bs, 2H).

### Intermediates 27-28

The following Intermediates were synthesized by an analogous method to Intermediate 11 from the starting materials (SM) given in the table below.

| Int | Compound | Data | SM |
|---|---|---|---|
| 27 | Ethyl 2-amino-4-(1-methyl-1H- imidazol-2-yl)-1,3-thiazole-5- carboxylate 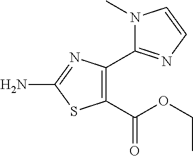 | MS (ES) (M + H)⁺: 253 for C₁₀H₁₂ClN₄O₂S NMR: 1.17 (t, 3H), 3.60 (s, 3H), 4.21 (m, 2H), 7.0 (s, 1H), 7.30 (s, 1H). | Intermediate 30 |
| 28 | Ethyl 2-amino-4-[1-(2- methoxyethyl)-1H-imidazol-2- yl]-1,3-thiazole-5-carboxylate 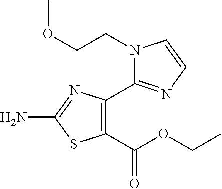 | MS (ES) (M + H)⁺: 297 for C₁₂H₁₆N₄O₃S NMR: 1.20 (t, 3H), 3.22 (s, 3H), 3.61 (m, 2H), 3.69 (s, 3H), 4.18 (m, 2H), 4.32 (m, 2H), 7.91 (s, 2H), 8.41 (s, 2H). | Intermediate 31 |

### Intermediate 29

### Ethyl 3-(1-methyl-1H-1,2,4-triazol-5-yl)-3-oxopropanoate

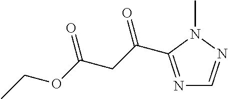

NaH (7.84 g, 196 mmol of a 60% dispersion in oil) was added portionwise to a solution of 6.18 g (34.5 mmol) of 1-(1-methyl-1H-1,2,4-triazol-5-yl)ethanone (Ohta, S.; Kawasaki, I.; Fukuno, A.; Yamashita, M.; Tada, T.; Kawabata, T. Chem. Pharm. Bull. (1993), 41(7), 1226-31) in 50 ml diethyl carbonate at 0° C. The mixture was heated to 90° C. for 2 hour forming thick slurry. After cooling to room temperature, the mixture was slowly transferred to 1N HCl over ice. The pH of the mixture was brought to about 7 with NaHCO₃ before being saturated with NaCl and extracted 4 times with EtOAc. The EtOAc was dried (Na₂SO₄) and concentrated to give oil that was chromatographed on silica gel (100% hexane followed by gradient elution to 40% EtOAc in hexane). Product (4.5 g) was obtained as a yellowish oily liquid. NMR: 1.30 (t, 3H), 4.11 (s, 2H), 4.27 (m, 5H), 7.96 (s, 1H).

### Intermediates 30-31

The following Intermediates were synthesized by an analogous method to Intermediate 14 from the starting materials (SM) given in the table below.

| Int | Compound | Data | SM |
|---|---|---|---|
| 30 | Ethyl 3-(1-methyl-1H-imidazol-2- yl)-3-oxopropanoate 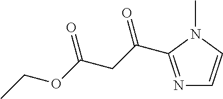 | MS (ES) (M + H)⁺: 197 for C₉H₁₂N₂O₃. NMR: 1.27 (t, 3H), 4.10 (s, 2H), 4.25 (m, 5H), 7.15 (s, 1H), 7.55 (s, 1H). | 1-(1-Methyl-1H- imidazol-2- yl)ethanone (Abarca-Gonzalez, B.; Jones, R. A.; Medio-Simon, M.; Quilez-Pardo, J.; Sepulveda-Arques, J.; Zaballos-Garcia, E. Synth. Comm. (1990), 20(3), 321- 31). |
| 31 | Ethyl 3-[1-(2-methoxyethyl)-1H- imidazol-2-yl]-3-oxopropanoate 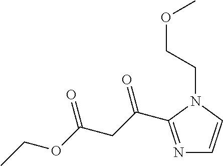 | MS (ES) (M + H)⁺: 241 for C₁₁H₁₆N₂O₄ NMR: 1.25 (t, 3H), 3.18 (s, 3H), 3.61 (m, 2H), 4.07 (s, 2H), 4.20 (m, 2H) 4.52 (m, 2H), 7.24 (s, 1H), 7.61 (s, 1H). | 1-[1-(2- Methoxyethyl)-1H- imidazol-2-yl] ethanone (WO200802022 A1) |

### Intermediate 32

### Ethyl (3S,4R)-4-{[(3-Bromo-4-chloro-5-methyl-1H-pyrrol-2-yl)carbonyl]amino}-3-methoxypiperidine-1-carboxylate

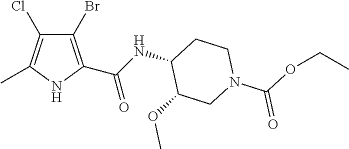

In a 50 ml round bottom flask 3-Bromo-4-chloro-5-methyl-1H-pyrrole-2-carboxylic acid (WO 2006087543, 8 gm 33.55 mmol) was dissolved in CH₂Cl₂ (100 ml) and DIEA (17.5 ml, 100.64 mmol), HATU (14.03, 36.9 mmol) was added and stirred for 5 minutes and then (3S,4R)-ethyl 4-amino-3-methoxypiperidine-1-carboxylate ((1R)-7,7-dimethyl-2-oxobicyclo[2.2.1]heptan-1-yl)methanesulfonate (WO2006087543, 16.04 mg, 36.9 mmol) was added portion wise and the resulting mixture was stirred for overnight at RT. The progress of the reaction was monitored through LCMS, which showed completion of the reaction after stirring the reaction mixture for overnight. The reaction mixture was diluted with DCM and washed with water. The organic layer was dried over sodium sulphate and concentrated under vacuum to give ethyl (3S,4R)-4-{[(3-Bromo-4-chloro-5-methyl-1H-pyrrol-2-yl)carbonyl]amino}-3-methoxypiperidine-1-carboxylate (12 gm).

MS (ES⁺): 423 for C₁₅H₂₁BrClN₃O₄

NMR: 1.22 (t, 3H), 1.70 (m, 2H), 2.20 (s, 3H), 2.97 (m, 2H), 3.32-3.43 (m, 4H), 3.90-4.30 (m, 5H), 7.25 (d, 1H), 12.19 (s, 1H).

### Intermediate 33-39

The following Intermediates were synthesized by an analogous method to Intermediate 17 from the starting materials (SM) given in the table below.

| Int | Compound | Data | SM |
|---|---|---|---|
| 33 | Ethyl (3S,4R)-4-{[(4-Bromo-3- chloro-5-methyl-1H-pyrrol-2- yl)carbonyl]amino}-3- methoxypiperidine-1-carboxylate 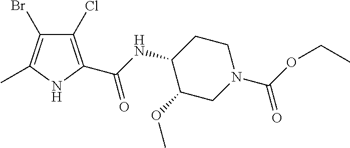 | 423 for C₁₅H₂₁BrClN₃O₄ NMR: 1.24 (t, 3H), 1.70 (m, 2H), 2.22 (s, 3H), 2.97 (m, 2H), 3.32-3.43 (m, 4H), 3.90-4.30 (m, 5H), 7.26 (d, 1H), 12.22 (s, 1H). | 4-Bromo-3-chloro- 5-methyl-1H- pyrrole-2- carboxylic acid and ethyl (3S,4R)- 4-amino-3- methoxypiperidine- 1-carboxylate (WO 2006087543 A1) |
| 34 | Ethyl (3S,4R)-4-{[(3,5-Dichloro- 5-methyl-1H-pyrrol-2- yl)carbonyl]amino}-3- methoxypiperidine-1-carboxylate 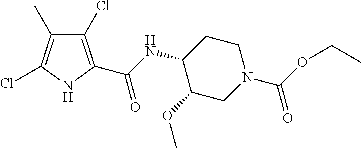 | MS (ES) (M − H)⁻: 379 for C₁₅H₂₁N₃O₄ NMR: 1.20 (t, 3H), 1.52 (m, 1H), 1.75 (m, 1H), 2.0 (s, 3H), 2.77-3.01 (m, 2H), 3.30-3.33 (m, 4H), 3.79- 4.33 (m, 5H), 6.80 (s, 1H), 7.55 (d, 1H), 12.0 (s, 1H). | 3,5-Dichloro-5- methyl-1H- pyrrole-2- carboxylic acid and ethyl (3S,4R)- 4-amino-3- methoxypiperidine- 1-carboxylate (WO 2006087543 A1) |
| 35 | Ethyl (3S,4R)-4-[(3-Bromo-4- cyano-5-methyl-1H-pyrrole-2- carbonyl)-amino]-3-methoxy- piperidine-1-carboxylate 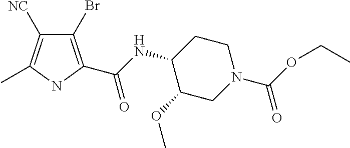 | MS (ES) (M + H)⁻: 414 for C₁₆H₂₁BrN₄O₄ | 3-Bromo-4-cyano- 5-methyl-1H- pyrrole-2- carboxylic acid and ethyl (3S,4R)- 4-amino-3- methoxypiperidine- 1-carboxylate (WO 2006087543 A1) |
| 36 | Ethyl (3S,4R)-4-[(4-Bromo-3- cyano-5-methyl-1H-pyrrole-2- carbonyl)-amino]-3-methoxy- piperidine-1-carboxylate 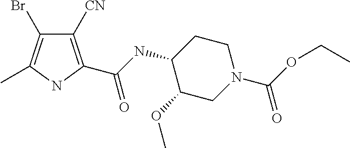 | MS (ES) (M + H)⁻: 414 for C₁₆H₂₁BrN₄O₄ | 3-Cyano-4- Bromo-5-methyl- 1H-pyrrole-2- carboxylic acid and ethyl (3S,4R)- 4-amino-3- methoxypiperidine-1- carboxylate (WO 2006087543 A1) |
| 37 | Ethyl (3S,4R)-4-[(3-Bromo-4- chloro-5-methyl-1H-pyrrole-2- carbonyl)-amino]-3-ethoxy- piperidine-1-carboxylate 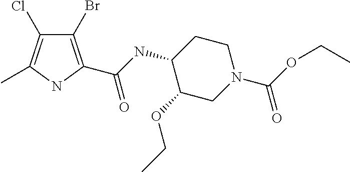 | MS (ES) (M + 2)⁻: 438 for C₁₆H₂₄BrClN₃O₄ | 3-Bromo-4- chloro-5-methyl- 1H-pyrrole-2- carboxylic acid (WO 2006087543 A1) and Intermediate 63 |
| 38 | Ethyl (3S,4R)-4-[(4-Bromo-3- chloro-5-methyl-1H-pyrrole-2- carbonyl)-amino]-3-ethoxy- piperidine-1-carboxylate 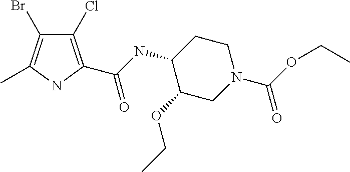 | MS (ES) (M + 2)⁺: 438.1 for C₁₆H₂₃BrClN₃O₄ | 4-Bromo-3- chloro-5-methyl- 1H-pyrrolo-2- carboxylic acid (WO 2006087543 A1) and Intermediate 63 |
| 39 | Ethyl (3S,4R)-4-[(3-Bromo-4- chloro-5-methyl-1H-pyrrole-2- carbonyl)-amino]-3- cyclopropylmethoxy-piperidine-1- carboxylale 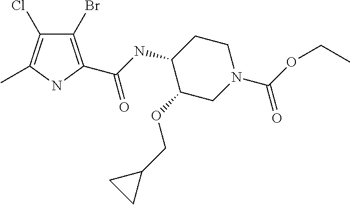 | MS (ES) (M + 2)⁺: 464 for C₁₈H₂₅BrClN₃O₄ | 3-Bromo-4- chloro-5-methyl- 1H-pyrrole-2- carboxylic acid (WO 2006087543 A1) and Intermediate 70 |

### Intermediate 40

### 3-Bromo-4-chloro-N-((3S,4R)-3-methoxypiperidin-4-yl)-5-methyl-1H-pyrrole-2-carboxamide hydrochloride

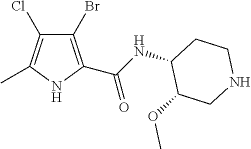

In a 250 mL round-bottomed flask (3S,4R)-ethyl 4-(3-bromo-4-chloro-5-methyl-1H-pyrrole-2-carboxamido)-3-methoxypiperidine-1-carboxylate (Intermediate 32, 12.00 g, 28.39 mmol) was dissolved in EtOH (100 mL). NaOH (10M solution) (14.76 g, 369 mmol in 40 ml of water) was then added and the reaction mixture was heated to 80° C. for 2 days. The progress of the reaction was monitored through LCMS. The reaction mixture was evaporated in vacuo, ice-cold water (25 ml) was added and the mixture was neutralized with 6N HCl (pH6), sonicated and the solid precipitate was filtered and dried under high vacuum afforded the product as pale brown solid (10 g).

MS (ES⁺): 351 for C₁₂H₁₇BrClN₃O₂

NMR: 1.62 (m, 2H), 2.30 (s, 3H), 2.61 (dm, 2H), 2.90 (dm, 1H), 3.14 (dm, 1H), 3.37 (m, 4H), 7.21 (d, 1H).

### Intermediates 41-47

The following Intermediates were synthesized by an analogous method to Intermediate 20 from the starting materials (SM) given in the table below.

| Int | Compound | Data | SM |
|---|---|---|---|
| 41 | 4-Bromo-3-chloro-N-((3S,4R)- 3-methoxypiperidin-4-yl)-5- methyl-1H-pyrrole-2- carboxamide hydrochloride 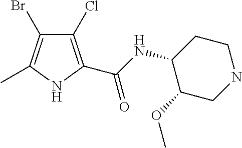 | MS (ES⁺): 351 for C₁₂H₁₇BrClN₃O₂ NMR: 1.80 (m, 2H), 2.20 (s, 3H), 2.61 (dm, 2H), 2.90 (dm, 1H), 3.14 (dm, 1H), 3.37 (m, 4H), 7.25 (d, 1H). | Intermediate 33 |
| 42 | 3,5-dichloro-N-((3S,4R)-3- methoxypiperidin-4-yl)-4-methyl- 1H-pyrrole-2-carboxamide hydrochloride 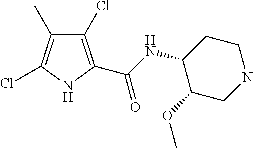 | MS (ES⁺): 306 for C₁₂H₁₇C₁₂N₃O₂ NMR: 1.65 (m, 2H), 1.95 (s, 3H), 2.63-2.73 (dm, 2H), 2.90 (dm, 1H), 3.20-3.40 (m, 5H), 4.15 (m, 1H) 7.26 (d, 1H). | Intermediate 34 |
| 43 | 3-Bromo-4-cyano-N-((3S,4R)-3- methoxypiperidin-4-yl)-5-methyl- 1H-pyrrole-2-carboxamide hydrochloride 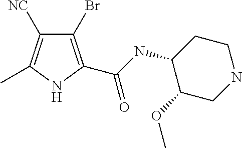 | MS (ES⁺): 342 for C₁₃H₁₇BrN₄O₂ | Intermediate 35 |
| 44 | 4-Bromo-3-cyano-N-((3S,4R)-3- methoxypiperidin-4-yl)-5-methyl- 1H-pyrrole-2-carboxamide hydrochloride 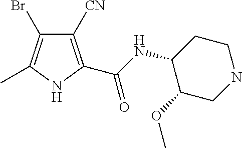 | MS (ES⁺): 342 for C₁₃H₁₇BrN₄O₂ | Intermediate 36 |
| 45 | 3-Bromo-4-chloro-N-((3S,4R)- 3-ethoxypiperidin-4-yl)-5-methyl- 1H-pyrrole-2-carboxamide hydrochloride 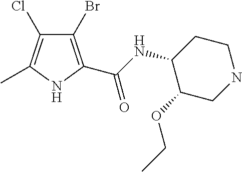 | MS (ES⁺): 366 for C₁₃H₁₉BrClN₃O₂ NMR: 1.15 (3H, t), 1.65 (2H, m), 2.2 (3H, s), 2.65 (2H, m), 2.93 (1H, dd), 3.15 (2H, m), 3.5 (2H, m), 4.15 (1H, m), 7.35 (1H, d). | Intermediate 37 |
| 46 | 4-Bromo-3-chloro-N-((3S,4R)- 3-ethoxypiperidin-4-yl)-5-methyl- 1H-pyrrole-2-carboxamide hydrochloride 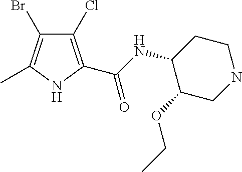 | MS (ES⁺): 366 for C₁₃H₁₉BrClN₃O₂ | Intermediate 38 |
| 47 | 3-Bromo-4-chloro-N-((3S,4R)- 3-cyclopropylmethoxypiperidin- 4-yl)-5-methyl-1H-pyrrole-2- carboxamido hydrochloride 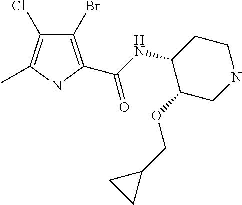 | MS (ES⁺): 392 for C₁₅H₂₁BrClN₃O₂ | Intermediate 39 |

### Intermediate 48

### Ethyl 5-Methyl-1H-pyrrole-2-carboxylate

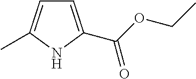

A solution of sodium nitrate (630 g, 9.230 moles) in water (1 L) was added dropwise to a mechanically stirred solution of ethyl acetoacetate (1 L) in glacial acetic acid (1.5 L) at 0° C. After 12 hrs the reaction mixture was treated with acetyl acetaldehyde dimethylacetal (1022 ml, 7.69 moles) and zinc dust (1106 g, 16.92 moles) was added portionwise for 8 hrs so that internal temperature should not rise above 60° C. The mixture was heated to 120° C. for 20 min and after cooling to 50° C., the reaction mixture was poured into ice water. Solid was filtered. The crude material was purified by column chromatography (100% hexane followed by gradient elution to 4% ethyl acetate in hexane). Product (227 g) was obtained as a pale yellow solid.

NMR (400 MHz, DMSO, δ): 1.24 (t, 3H), 2.18 (s, 3H), 4.17 (q, 2H), 5.84 (s, 1H), 6.63 (s, 1H), 11.55 (brs, 1H).

MS (ES) (M+H)⁺: 154 for C₈H₁₁NO₂

### Intermediate 49

### Ethyl 4-Bromo-5-methyl-1H-pyrrole-2-carboxylate

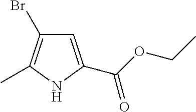

### Intermediate 50

### Ethyl 3,4-dibromo-5-methyl-1H-pyrrole-2-carboxylate

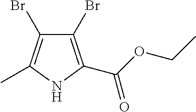

N-bromo succinimide (250 g, 1.410 moles) was added portionwise to a solution of ethyl 5-methyl-1-H-pyrrole-2-carboxylate (180 g, 1.175 moles) in chloroform (2 L) for 7 hrs at RT. The mixture was heated to 50° C. for 5 hrs. After cooling to room temperature the reaction mixture was concentrated and the mixtures of bromo compounds were separated by column chromatography. Intermediate 49 (Ethyl 4-Bromo-5-methyl-1H-pyrrole-2-carboxylate, 200 g) was eluted with 2% ethyl acetate in hexane as off-white solid. NMR (400 MHz, DMSO, δ): 1.26 (t, 3H), 2.17 (s, 3H), 4.21 (q, 2H), 6.73 (s, 1H), 12.08 (s, 1H). MS (ES) (M+H)⁺: 233 for C₈H₁₀BrNO₂. Intermediate 50 (Ethyl 3,4-dibromo-5-methyl-1H-pyrrole-2-carboxylate 60 g) was eluted with 6% ethyl acetate in hexane as brown solid. NMR (400 MHz, DMSO, δ): 1.27 (t, 3H), 2.21 (s, 3H), 4.24 (q, 2H), 12.39 (s, 1H). MS (ES) (M+H)⁺: 311 for C₈H₉Br₂NO₂

### Intermediate 51

### Ethyl 4-bromo-3-cyano-5-methyl-1H-pyrrole-2-carboxylate

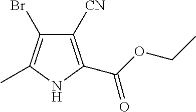

Copper(II) cyanide (69.1 g, 0.776 moles) was added to a solution of ethyl 3,4-dibromo-5-methyl-1H-pyrrole carboxylate (60 g, 0.194 moles) in DMF (600 ml). The mixture was heated to 125° C. for overnight. After cooling to room temperature the reaction mixture was diluted with water, filtered through celite and washed with ethyl acetate. The filtrate was extracted four times with ethyl acetate. The combined organic layer were dried over sodium sulphate and concentrated. The crude material was purified by column chromatography (100% hexane followed by gradient elution to 10% ethyl acetate in hexane). Product (13 g) was obtained as off white solid.

NMR (400 MHz, DMSO, δ): 1.29 (t, 3H), 2.19 (s, 3H), 4.30 (q, 2H), 13.10 (s, 1H).

MS (ES) (M+H)⁺: 258 for C₉H₉BrN₂O₂

### Intermediate 52

### 4-Bromo-3-cyano-5-methyl-1H-pyrrole-2-carboxylic acid

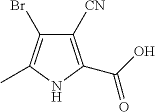

Ethyl 4-bromo-3-cyano-5-methyl-1H-pyrrole-2-carboxylate (13 g, 0.0505 moles) was dissolved in mixture of MeOH:THF:H₂O (100 ml each). Lithium hydroxide (32.5 g, 0.758 moles) was added and heated to 60° C. for overnight. After removing the solvents, the crude mixture was acidified with conc. HCl and extracted with ethyl acetate four times. The combined organic layers were dried over sodium sulfate and concentrated. Product (10.3 g) was obtained as white solid.

NMR (400 MHz, DMSO, δ): 2.19 (s, 3H), 12.98 (s, 1H), 13.72 (brs, 1H).

MS (ES) (M+H)⁺: 230 for C₇H₅BrN₂O₂

### Intermediate 53

### Ethyl 4-Cyano-5-methyl-1H-pyrrole-2-carboxylate

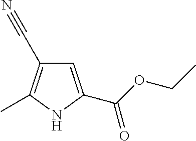

Copper(II) cyanide (77.07 g, 0.866 moles) was added to a solution of ethyl 4-Bromo-5-methyl-1H-pyrrole-2-carboxylate (50 g, 0.216 moles) in DMF (500 ml). The mixture was heated to 150° C. for overnight. After cooling to room temperature the reaction mixture was diluted with water, filtered through celite and washed with ethyl acetate. The filtrate was extracted four times with ethyl acetate. The combined organic layer were dried over sodium sulphate and concentrated. The crude material was purified by column chromatography (100% hexane followed by gradient elution to 10% ethyl acetate in hexane). Product (45 g) was obtained as off white solid.

NMR (400 MHz, DMSO, δ): 1.25 (t, 3H), 2.32 (s, 3H), 4.23 (q, 2H), 7.06 (s, 1H), 12.58 (s, 1H).

MS (ES) (M+H)⁺: 179 for C₉H₁₀N₂O₂

### Intermediate 54

### Ethyl 3-Bromo-4-cyano-5-methyl-1H-pyrrole-2-carboxylate

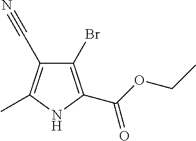

Bromine (13.73 ml, 0.266 moles) was dropwise to a solution of ethyl 4-cyano-5-methyl-1H-pyrrole-2-carboxylate (45 g, 0.254 moles) in acetic acid (400 ml) at 0° C. The reaction mixture was allowed to warm to RT and stirred for 2 hrs. The progress of the reaction was monitored by NMR. After completion of the reaction, acetic acid was removed under reduced pressure and the resulting solid was washed with hexane to afford to the product as light brown solid. (60 g)

NMR (400 MHz, DMSO, δ): 1.30 (t, 3H), 2.36 (s, 3H), 4.28 (q, 2H), 12.85 (s, 1H).

MS (ES) (M+H)⁺: 258 for C₉H₉BrN₂O₂

### Intermediate 55

### 3-Bromo-4-cyano-5-methyl-1H-pyrrole-2-carboxylic acid

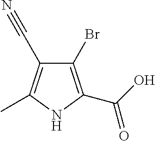

Ethyl 3-Bromo-4-cyano-5-methyl-1H-pyrrole-2-carboxylate (60 g, 0.233 moles) was dissolved in mixture of MeOH:THF:H₂O (200 ml each). Lithium hydroxide (146.9 g, 3.501 moles) was added and heated to 60° C. for overnight. After removing the solvents, the crude mixture was acidified with conc. HCl and extracted with ethyl acetate four times. The combined organic layers were dried over sodium sulfate and concentrated. Product (40 g) was obtained as off-white solid.

NMR (400 MHz, DMSO, δ): 2.33 (s, 3H), 12.76 (s, 1H), 13.32 (brs, 1H).

MS (ES) (M+H)⁺: 230 for C₇H₅BrN₂O₂

### Intermediate 56

### 3-Hydroxy-4,4-dimethoxypiperidine-1-carboxylic acid ethyl ester

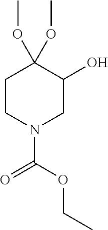

To the solution of 4-Oxopiperidine-1-carboxylic acid ethyl ester (300 g, 1.752 moles) in dry methanol (1 L), cooled solution of KOH (422.7 g, 7.535 moles) in dry Methanol (1.2 L) was added drop wise at 0° C., After the addition iodobenzene diacetate (846.6 g, 2.628 moles) was added in portions at 0° C. to 5° C. RM was stirred at 0° C. for 30 minutes and stirred at RT overnight. The progress of the reaction was monitored by TLC. After completion of the reaction, RM was concentrated directly to remove the methanol. The residue was dissolved in water (3 L) and extracted with ethyl acetate (2×2 L). The combined organic layers were washed with water (1.5 L), brine solution (200 ml) and dried over sodium sulphate. Purification of the crude product by column chromatography (25% Ethyl acetate in Pet ether) gave the desired product as light yellow viscous liquid (410 g).

¹H NMR (400 MHz, CDCl₃): δ 4.12 (q, 2H), 4.09 (bs, 2H), 3.77 (bs, 1H), 3.25 (S, 6H), 3.15 (bs, 1H), 2.89 (bs, 1H), 2.04 (bs, 1H), 1.87-1.71 (m, 2H), 1.27 (t, 3H).

### Intermediate 57

### 3-Ethoxy-4,4-Dimethoxypiperidine-1-carboxylic acid ethyl ester

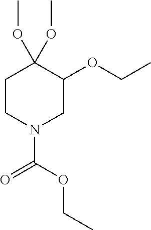

To the suspension of Sodium hydride (76.5 g, 1.5947 moles) in dry THF (600 ml), a solution of 3-Hydroxy-4,4-dimethoxypiperidine-1-carboxylic acid ethyl ester (310 g, 1.328 moles) in dry THF (500 ml) was added drop wise at 0° C. After the addition, RM was stirred at RT for 2 hrs and then stirred at 50° C. overnight. The reaction was monitored by TLC, reaction was not completed, about 20% of starting material observed in TLC. The reaction was stirred at 50° C. overnight. RM was quenched with water (100 ml) slowly at 0° C., then diluted with water (500 ml) and extracted with ethyl acetate (3×800 ml). The combined ethyl acetate layers were washed with brine solution (250 ml) and dried over sodium sulfate. Crude product was purified by column chromatography (20% Ethyl acetate in Pet ether). Product was obtained as pale yellow viscous liquid (188 g). 58 g of the unreacted starting material was recovered.

¹H NMR (400 MHz, CDCl₃): δ 4.15 (bs, 1H), 4.13 (q, 2H), 4.10-3.98 (bs, 1H), 3.44 (q, 2H), 3.23 (S, 6H), 3.02 (bs, 1H), 2.98-2.82 (bs, 1H), 2.91-2.73 (bs, 2H), 1.27 (t, 3H), 1.24 (t, 3H).

### Intermediate 58

### 3-Ethoxy-4-oxo-piperidine-1-carboxylic acid ethyl ester

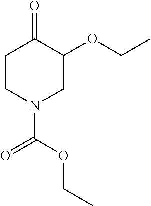

To the solution of 3-Ethoxy-4,4-Dimethoxypiperidine-1-carboxylic acid ethyl ester (140 g, 0.566 moles) in THF (350 ml), 5% v/v aq. sulfuric acid (253 ml, 0.238 moles) was added drop wise and the reaction was heated to 60° C. overnight. The reaction was monitored by TLC and RM was concentrated to remove THF. The residue was dissolved in water (300 ml) and the P^{H} of the solution was adjusted to 10 using solid sodium bicarbonate, then extracted with ethyl acetate (3×400 ml). The combined ethyl acetate layers were washed with water (200 ml), brine solution (100 ml), dried over sodium sulfate and evaporated to dryness offered pure product as light yellow liquid (107 g).

¹H NMR (300 MHz, CDCl₃): δ 4.23 (bs, 1H), 4.19 (q, 2H), 4.11 (bs, 2H), 3.80 (bs, 1H), 3.72 (q, 2H), 3.60 (bs, 1H), 3.37 (bs, 2H), 2.50 (bs, 1H), 2.44 (bs, 1H), 1.31 (t, 3H), 1.22 (bs, 1H).

### Intermediate 59

### Cis (+) 3-Ethoxy-4-(1-phenyl-ethylamino)-piperidine-1-carboxylic acid ethyl ester

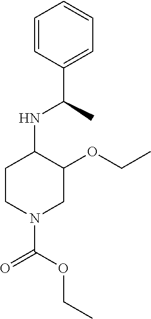

R (+)-α-methyl benzyl amine (72 ml, 0.558 moles) in freshly distilled THF (200 ml) was added drop wise to the solution of 3-Ethoxy-4-oxo-piperidine-1-carboxylic acid ethyl ester (100 g, 0.465 moles) in freshly distilled THF (600 ml). RM was stirred for 1 hr and sodium triacetoxyborohydride (108.4 g, 0.511 moles) was added portion wise at 0° C. The stirring was continued at 0° C. for 4 hrs and then stirred at RT for overnight. The reaction was monitored by TLC. The RM was quenched with water (150 ml) at 0° C., then basified to P^{H} 8 using solid sodium bicarbonate and extracted with ethyl acetate (3×600 ml). The combined organic layers were washed with water (100 ml), brine solution (100 ml) and dried over sodium sulfate. Crude product was purified by column chromatography (30% Ethyl acetate in pet ether). Product was obtained as pale yellow viscous oil (54 g).

¹H NMR (400 MHz, CDCl₃): δ 7.38-7.33 (m, 5H), 5.10 (S, 1H), 4.12 (q, 2H), 4.02 (bs, 1H), 3.80 (bs, 2H), 3.62 (bs, 1H), 3.43 (q, 1H), 3.34 (q, 1H), 2.81-2.62 (bs, 2H), 1.69-1.63 (bs, 4H), 1.28-1.22 (m, 6H).

MS (ES) (M+H)⁺: 321.2 for C₁₈H₂₈N₂O₃

### Intermediate 60

### Cis (+) 4-Amino-3-ethoxy-piperidine-1-carboxylic acid ethyl ester

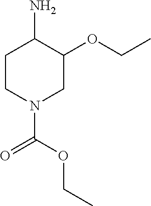

5% Pd—C (3 g) (Aldrich) was added to the solution of Cis 3-Ethoxy-4-(1-phenyl-ethylamino)-piperidine-1-carboxylic acid ethyl ester (32 g, 0.099 moles) in dry methanol (350 ml) and the resulting reaction mixture was hydrogenated (3 Kg/cm3 hydrogen pressure) in an autoclave (1000 ml capacity) at 50° C. overnight. The reaction was monitored by TLC. RM was filtered through celite bed, washed with methanol (150 ml) and concentrated under vacuum offered the product as light yellowish oil (19 g).

¹H NMR (400 MHz, CDCl₃): δ 4.52-4.27 (bs, 1H), 4.22-4.17 (bs, 1H), 4.11 (q, 2H), 3.73-3.68 (bs, 2H), 3.42-3.35 (m, 2H), 2.86-2.80 (bs, 2H), 1.86-1.83 (bs, 2H), 1.32 (t, 3H), 1.18 (t, 3H).

MS (ELSD) (M+H)⁺: 217.2 for C₁₀H₂₀N₂O₃

### Intermediate 61

### Cis (+) 4-Benzyloxycarbonyl amino-3-ethoxy-piperidine-1-carboxylic acid ethyl ester

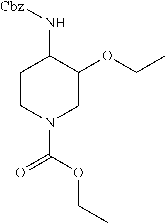

Benzyloxy chloroformate (95% solution in toluene) (22.4 g, 0.131 moles) was added slowly the solution of Cis 4-Amino-3-ethoxy-piperidine-1-carboxylic acid ethyl ester (19 g, 0.0878 moles) in saturated sodium bicarbonate solution (110 ml) at 0° C. and then stirred at RT overnight. The reaction was monitored by TLC. The RM was extracted with ethyl acetate (2×300 ml), the combined organic layers were washed with water (100 ml), brine solution (50 ml) and dried over sodium sulfate. Purification by column chromatography (25% Ethyl acetate in Pet ether) offered the pure product as pale yellow viscous liquid (24 g).

¹H NMR (300 MHz, CDCl₃) δ 7.38-7.32 (m, 5H), 5.30-5.23 (b, 1H), 5.15 (S, 2H), 4.40-4.29 (b, 1H), 4.13 (t, 2H), 3.742H), 3.38 (b, 1H), 3.32 (q, 2H), 2.96-2.78 (b, 2H), 1.76 (b, 2H), 1.28 (t, 3H), 1.25 (t, 3H).

MS (ES) (M+H)⁺: 351.0 for C₁₈H₂₈N₂O₃

### Intermediate 62

### (3S,4R) 4-Benzyloxycarbonyl amino-3-ethoxy-piperidine-1-carboxylic acid ethyl ester

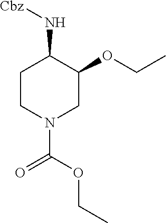

Chiral chromatography intermediate 61 (28 gm) using the following conditions afforded the title compound (18 g) pure major isomer as pale yellow oil

Method Information:
Column: CHIRAL PAK AD-H (4.5 mm×250 nm×5μ)
Flow rate: 1.0 ml/min
Mobile phase:
   A: 0.2% Diethyl amine in Hexane
   B: Ethanol (10): Methanol (10)
Injection vol. 10.0 μl
Retention time (RT):
   Major isomer: 7.78
   Minor isomer: 5.85

¹H NMR (400 MHz, CDCl₃): δ 7.38-7.32 (m, 5H), 5.24 (bs, 1H), 5.11 (S, 2H), 4.50-4.27 (bs, 1H), 4.15-4.09 (m, 3H), 3.75-3.67 (2H), 3.46-3.29 (bs, 2H), 2.82-2.79 (bs, 2H), 1.76-1.63 (bs, 2H), 1.37 (t, 3H), 1.26 (t, 3H).

MS (ES) (M+H)⁺: 351.2 for C₁₈H₂₈N₂O₃

### Intermediate 63

### (3S,4R)-4-Amino-3-ethoxy-piperidine-1-carboxylic acid ethyl ester

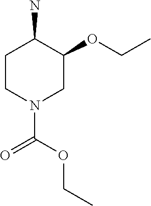

10% Pd—C (1.5 g) was added to the solution of (3S,4R)-4-Benzyloxycarbonyl amino-3-ethoxy-piperidine-1-carboxylic acid ethyl ester (12 g, 0.032 moles) in dry methanol and hydrogenated (3 Kg/cm³ hydrogen pressure) in Parr shaker at RT. The reaction was monitored by TLC. The reaction mixture was filtered through celite bed, washed with methanol (100 ml) and concentrated to dryness offered pure product as a hygroscopic white solid (8.4 g)

¹H NMR (400 MHz, CDCl₃): δ 4.14-4.09 (m, 3H), 4.08-4.38 (bs, 1H), 3.78-3.63 (bs, 1H), 3.47-3.38 (m, 2H), 2.99-2.29 (bs, 2H), 2.76 (bs, 2H), 1.78-1.59 (m, 2H), 1.25 (t, 3H), 1.18 (t, 3H)

MS (ELSD) (M+H)⁺: 217.2 for C₁₀H₂₀N₂O₃.

### Intermediate 64

### 3-Cyclopropylmethoxy-4,4-dimethoxypiperidine-1-carboxylic acid ethyl ester

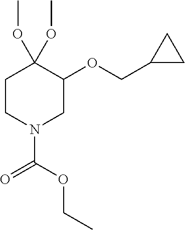

To the suspension of Sodium hydride (24.6 g, 0.5144 moles) in dry THF (400 ml), a solution of 3-hydroxy-4,4-dimethoxypiperidine-1-carboxylic acid ethyl ester (100 g, 0.4287 moles) in dry THF (300 ml) was added drop wise at 0° C. After the addition, RM was stirred at RT for 2 hrs, and then stirred at 50° C. overnight. Reaction was monitored by TLC, reaction was not completed, about 40% of starting material observed in TLC. Reaction was stirred at 50° C. overnight. RM was quenched with water (50 ml) slowly at 0° C., then diluted with water (300 ml) and extracted with ethyl acetate (3×400 ml). The combined ethyl acetate layers were washed with brine solution (250 ml) and dried over sodium sulfate. Crude product was purified by column chromatography (15% Ethyl acetate in Pet ether). Product was obtained as pale yellow viscous liquid (68 g). 15 g of the unreacted starting material was recovered.

¹H NMR (400 MHz, CDCl₃): δ 4.26-4.11 (Bs, 1H), 4.09 (q, 2H), 4.05-3.88 (bs, 1H), 3.50-3.42 (bs, 2H), 3.30 (bs, 1H), 3.24 (S, 6H), 3.06-2.97 (Bs, 1H), 2.86-2.81 (bs, 1H), 1.84-1.68 (b, 2H), 1.27 (t, 3H), 1.05 (m, 1H), 0.512H), 0.20 (bs, 2H)

### Intermediate 65

### 3-Cyclopropylmethoxy-4-oxo-piperidine-1-carboxylic acid ethyl ester

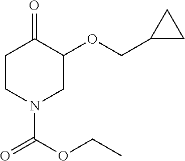

To the solution of 3-Cyclopropylmethoxy-4,4-dimethoxypiperidine-1-carboxylic acid ethyl ester (68 g, 0.236 moles) in THF (250 ml), 5% v/v aq. sulfuric acid (253 ml) was added drop wise and the reaction was heated to 60° C. overnight. Reaction was monitored by TLC. RM was concentrated directly to remove THF and the residue was dissolved in water (200 ml) and the P^{H} of the solution was adjusted to 10 using solid sodium bicarbonate then extracted with ethyl acetate (3×300 ml). The combined ethyl acetate layer was washed with water (200 ml), brine solution (100 ml), dried over sodium sulfate and evaporated to dryness to affor pure product as light yellow liquid (52 g).

¹H NMR (300 MHz, CDCl₃): δ 4.09 (m, 2H), 3.87 (bs, 1H), 3.50 (m, 1H), 3.36 (bs, 2H), 2.60-2.55 (bs, 1H), 2.47-2.39 (bs, 1H), 1.36 (t, 3H), 1.05 (m, 1H), 0.58 (bs, 2H), 0.21 (bs, 2H).

### Intermediate 66

### Cis (+) 3-Cyclopropylmethoxy-4-(1-phenyl-ethylamino)-piperidine-1-carboxylic acid ethyl ester

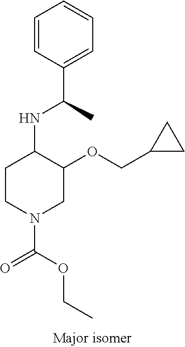

R (+)-α-methyl benzyl amine (31.46 g, 0.259 moles) in freshly distilled THF (200 ml) was added drop wise to the solution of 3-Cyclopropylmethoxy-4-oxo-piperidine-1-carboxylic acid ethyl ester (52 g, 0.216 moles) in freshly distilled THF (400 ml). RM was stirred at this temperature for another 60 minutes and sodium triacetoxyborohydride (45.86 g, 0.2166 moles) was added portion wise at 0° C. and stirred at 0° C. for 4 hrs, then stirred at RT overnight. The progress of the reaction was monitored by TLC. The RM was quenched with water (100 ml) at 0° C., then basified to P^{H} 8 using solid sodium bicarbonate and extracted with ethyl acetate (3×400 ml). The combined organic layers were washed with water (200 ml), brine solution (100 ml) and dried over sodium sulfate. Crude product was purified by column chromatography (25% Ethyl acetate in pet ether). Product was obtained as pale yellow viscous oil (56 g).

¹H NMR (400 MHz, CDCl₃): δ 7.40-7.30 (m, 5H), 4.22-4.16 (bs, 1H), 4.07 (q, 2H), 3.64 (bs, 1H), 3.54 (bs, 1H), 3.48 (bs, 1H), 2.96 (bs, 1H), 2.70-2.55 (bs, 3H), 1.65 (bs, 2H), 1.36-1.34 (bs, 3H), 1.26 (t, 3H), 1.09 (bs, 1H), 0.61-0.47 (bs, 2H), 0.28-0.14 (bs, 2H)

MS (ES) (M+H)⁺: 347.1 for C₂₀H₃₀N₂O₃

### Intermediate 67

### Cis (+) 4-Amino-3-cyclopropylmethoxy-piperidine-1-carboxylic acid ethyl ester

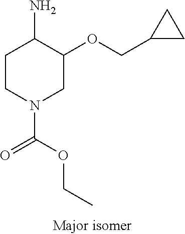

10% Pd—C (8 g) (Aldrich) was added to the solution of C is 3-Cyclopropylmethoxy-4-(1-phenyl-ethylamino)-piperidine-1-carboxylic acid ethyl ester (58 g, 0.1675 moles) in dry methanol (450 ml). Ammonium formate (74 g, 1.172 moles) was added at once and the resulting reaction mixture was heated in an autoclave (1000 ml capacity) at 65° C. overnight. Reaction was monitored by TLC. After two days, RM was filtered through celite bed, washed with methanol (450 ml) and concentrated under vacuum. The resulting white paste, chloroform (500 ml) was added and the insoluble materials were filtered off. The concentration of the filtrate offered product as light yellowish oil (29 g).

¹H NMR (400 MHz, CDCl₃): δ 4.44-4.30 (bs, 2H), 4.14 (q, 2H), 3.75 (bs, 3H), 3.49-3.35 (bs, 3H), 2.88-2.78 (bs, 2H), 1.96-1.86 (bs, 4H), 1.26 (t, 3H), 1.0-6 (m, 1H), 0.52 (b, 2H), 0.22 (bs, 2H).

MS (ELSD) (M+H)⁺: 243.2 for C₁₂H₂₂N₂O₃

### Intermediate 68

### Cis (+) 4-Benzyloxycarbonyl amino-3-cyclopropylmethoxy-piperidine-1-carboxylic acid ethyl ester

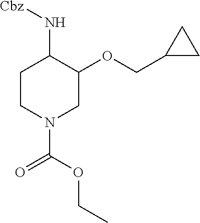

Benzyloxy chloroformate (95% solution in toluene) (15.3 g, 0.0897 moles) was added slowly to the solution of Cis 4-Amino-3-cyclopropylmethoxy-piperidine-1-carboxylic acid ethyl ester (16 g, 0.055 moles) in saturated sodium bicarbonate solution (200 ml) at 0° C. and stirred at RT overnight. Reaction was monitored by TLC, RM was extracted with ethyl acetate (2×250 ml). The combined organic layers were washed with water (100 ml), brine solution (50 ml) and dried over sodium sulfate. Purification by column chromatography (20% Ethyl acetate in Pet ether) offered the pure product as pale yellow viscous liquid (24 g).

¹H NMR (300 MHz, CDCl₃) δ 7.37-7.32 (bs, 5H), 5.32-5.21 (b, 1H), 5.11 (S, 2H), 4.44-4.27 (bs, 1H), 4.11 (q, 2H), 3.75 (bs, 1H), 3.48 (bs, 1H), 3.38 (m, 1H), 3.25 (b, 1H), 2.79 (bs, 2H), 1.76-1.71 (bs, 2H), 1.26 (t, 3H), 0.98 (m, 1H), 0.48 (bs, 2H), 0.17 (bs, 2H).

MS (ES) (M+H)⁺: 376.9 for C₂₀H₂₈N₂O₅

### Intermediate 69

### (3S,4R) 4-Benzyloxycarbonyl amino-3-cyclopropylmethoxy-piperidine-1-carboxylic acid ethyl ester

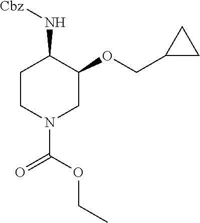

Chiral chromatography of intermediate 68 (24 gm) using the following conditions afforded the title compound (8.5 g) pure major isomer as pale yellow oil

### Method Information

Column: CHIRAL PAK AD-H (250%4.6), 5μ, SC/693.
Flow rate: 1.0 ml/min
Mobile phase:
   A: 0.2% Diethyl amine in Hexane
   B: Ethanol (10): Methanol (10)
Injection vol. 50.0 μl
Retention time (RT):
   Major isomer: 10.58
   Minor isomer: 6.69

¹H NMR (400 MHz, CDCl₃): δ 7.38-7.33 (m, 5H), 5.31 (bs, 1H), 5.11 (S, 2H), 4.44-4.23 (bs, 1H), 4.11 (m, 3H), 3.76 (bs, 1H), 3.52 (bs, 1H), 3.40 (bs, 1H), 3.25 (bs, 1H), 2.87-2.74 (bs, 2H), 1.79-1.68 (bs, 2H), 1.26 (t, 3H), 1.02 (m, 1H), 0.49 (bs, 2H), 0.17 (b, 2H)

MS (ES) (M+H)⁺: 376.9 for C₂₀H₂₈N₂O₅

### Intermediate 70

### (3S,4R)-ethyl 4-amino-3-(cyclopropylmethoxy)piperidine-1-carboxylate

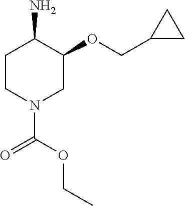

10% Pd—C (2.35 g) was added to the solution of (3S,4R)-4-Benzyloxycarbonyl amino-3-ethoxy-piperidine-1-carboxylic acid ethyl ester (8 g, 22.07 mmoles) in dry methanol and hydrogenated (3 Kg/cm³ hydrogen pressure) in Parr shaker at RT. The reaction was monitored by TLC. The reaction mixture was filtered through celite bed, washed with methanol (100 ml) and concentrated to dryness offered pure product as a hygroscopic white solid (5 g, 93%)

MS (ES) (M+H)⁺: 243 for C12H22N2O3

¹H NMR (400 MHz, DMSO-d₆) 0.01 (br. s., 2H) 0.28 (d, J=7.53 Hz, 2H) 0.75-0.91 (m, 1H) 1.27 (d, J=5.52 Hz, 2H), 2.34 (s, 1H), 2.60-2.74 (m, 2H), 2.74-2.87 (m, 2H), 3.14 (br. s., 2H), 3.83 (q, J=7.03 Hz, 2H).

### Intermediate 71

### 1-Ethyl-1H-1,2,4-triazole

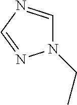

A solution of DBU (220.2 g, 1.736 moles) in THF (200 ml) was added dropwise to a mechanically stirred suspension of 1,2,4-triazole (100 g, 1.44 moles) and ethyl iodide (318 g, 2.026 moles) in dry THF (1 L) at 0° C., over a period of 3 hrs via addition funnel. The reaction mixture was allowed to warm to RT and stirred for overnight. The reaction mixture was filtered through celite and washed with THF (2×250 ml). The combined filtrates were concentrated and the residue was distilled under reduced pressure to give the product as colorless liquid. (55 gm)

NMR (400 MHz, CDCl3, δ): 1.51 (t, 3H), 4.21 (q, 4H), 7.92 (s, 1H), 8.05 (s, 1H).

### Intermediate 72

### 1-(2-Ethyl-2H-[1,2,4]triazol-3-yl)-ethanone

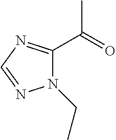

n-Butyl lithium (424 ml, 1.6M solution in THF, 0.679 moles) was added dropwise to a solution of 1-ethyl-1H-1,2,4-triazole (55 g, 0.566 moles) in THF (400 ml) at 0° C. After stirring for 1 hr at 0° C., N,N-dimethylacetamide (63 ml, 0.679 moles) was added to the reaction mixture and the stirring was continued for 1 hr. The progress of the reaction was monitored by TLC. The reaction mixture was quenched with sat. ammonium chloride solution and extracted with DCM. The combined organic layers were dried over sodium sulfate and concentrated. The crude material was purified by column chromatography (100% hexane followed by gradient elution to 10% ethyl acetate in hexane). Product (70 g) was obtained as light yellowish liquid. (Note: Since the product is volatile, it has to be distilled at less than 40° C.)

NMR (400 MHz, CDCl3, δ): 1.42 (t, 3H), 2.69 (s, 3H), 4.58 (q, 4H), 7.90 (s, 1H).

MS (ES) (M+H)⁺: 140 for C₆H₉N₃O.

### Intermediate 73

### 3-(2-Ethyl-2H-[1,2,4]triazol-3-yl)-3-oxo-propionic acid ethyl ester

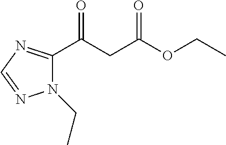

NaH (48.2 g, 2.012 moles of a 60% dispersion in oil) was added portionwise to a solution of 1-(2-Ethyl-2H-[1,2,4]triazol-3-yl)-ethanone (70 g, 0.53 moles) in 600 ml diethyl carbonate at 0° C. The mixture stirred at 0° C. for 1 hr and then heated to 60° C. for 2 hrs. The progress of the reaction was monitored by LCMS. After cooling to −10° C. by using ice-salt mixture, 6N HCl was added slowly to the reaction mixture by using additional funnel. The pH of the mixture was brought to about 7 with NaHCO₃ and extracted 4 times with EtOAc. The EtOAc was dried (Na₂SO₄) and concentrated to give oil that was chromatographed on silica gel (100% hexane followed by gradient elution to 40% EtOAc in hexane). Product (32.4 g) was obtained as a yellowish oily liquid.

NMR (400 MHz, DMSO, δ): 1.17 (t, 3H), 1.30 (t, 3H), 4.07 (q, 2H), 4.12 (s, 2H), 4.51 (q, 2H), 8.15 (s, 1H).

MS (ES) (M+H)⁺: 212 for C₉H₁₃N₃O₃

### Intermediate 74

### 2-Amino-4-(2-ethyl-2H-[1,2,4]triazol-3-yl)-thiazole-5-carboxylic acid ethyl ester

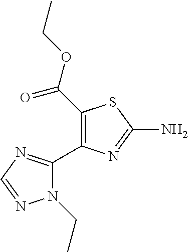

Sulfuryl chloride (14.13 ml, 0.175 moles) was added dropwise through the dropping funnel over a period of 30 min to a solution of 3-(2-Ethyl-2H-[1,2,4]triazol-3-yl)-3-oxo-propionic acid ethyl ester (32 g, 0.152 moles) in 1 L of DCM at 0° C. The reaction mixture was stirred for overnight at RT and quenched with aqueous sodium bicarbonate solution. The organic layer was separated and dried over sodium sulfate and concentrated. The residue was purified by silica gel column chromatography using 10% ethyl acetate in hexane.

To a solution of above chloro compound in ethanol (500 ml), thiourea (9.15 g, 0.12 moles) was added and refluxed for 6 hrs. The progress of the reaction was monitored by TLC. After completion of the reaction, the reaction mixture was cooled to RT and concentrated under reduced pressure. Ice-cooled water was added to the residue and neutralized with saturated sodium carbonate solution (200 ml). The precipitated solid was filtered and washed with water, ether and dried under high vacuum to afford the product as white solid. (24.3 g).

NMR (400 MHz, DMSO, δ): 1.03 (t, 3H), 1.25 (t, 3H), 4.00 (m, 4H), 7.98 (s, 1H), 8.07 (bs, 2H).

MS (ES) (M+H)⁺: 268 for C₁₀H₁₃N₅O₂S.

### Intermediate 75

### 2-Chloro-4-(2-ethyl-2H-[1,2,4]triazol-3-yl)-thiazole-5-carboxylic acid ethyl ester

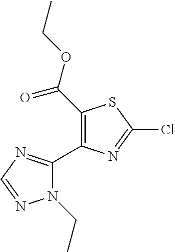

2-Amino-4-(2-ethyl-2H-[1,2,4]triazol-3-yl)-thiazole-5-carboxylic acid ethyl ester (24.3 g, 0.09 moles) was added to a mixture of acetic acid (150 ml) and conc. HCl (150 ml) at 0 to −5° C. Sodium nitrite (17.57 g, 0.254 moles) in water (100 ml) was added dropwise to above mixture. After complete addition, the reaction mixture was stirred for 1 hr at 0° C. Then urea (8.1 g, 0.135 moles) in water (100 ml) was added very slowly and stirring was continued for another 1 hr. The progress of the reaction was monitored by TLC. After completion of the reaction, ice cooled water was added, basified with sodium bicarbonate and extracted with ethyl acetate. The organic layer was separated, dried over sodium sulfate and concentrated. The residue was purified by silica gel column chromatography using 30% ethyl acetate in hexane to afford the product. (10.3 g).

NMR (400 MHz, DMSO, δ): 1.12 (t, 3H), 1.28 (t, 3H), 4.08 (q, 2H), 4.19 (q, 2H), 8.10 (s, 1H).

MS (ES) (M+H)⁺: 287 for C₁₀H₁₁ClN₄O₂S.

### Intermediate 76

### 1-Cyclopropylmethyl-1H-[1,2,4]triazole

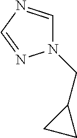

A solution of DBU (105.9 g, 0.695 moles) in THF (100 ml) was added dropwise to a mechanically stirred suspension of 1,2,4-triazole (40 g, 0.579 moles) and cyclopropylmethyl bromide (101.7 g, 0.753 moles) in dry THF (300 ml) at 0° C., over a period of 3 hrs via addition funnel. The reaction mixture was allowed to warm to RT and stirred for overnight. The reaction mixture was filtered through celite and washed with THF (2×250 ml). The combined filtrates were concentrated and the residue was distilled under reduced pressure to give the product as colorless liquid. (58 g)

NMR (400 MHz, CDCl3, δ): 0.41 (q, 2H), 0.69 (q, 2H), 1.3 (m, 1H), 4.02 (d, 2H), 7.93 (s, 1H), 8.15 (s, 1H).

MS (ES) (M+H)⁺: 124 for C₆H₉N₃.

### Intermediate 77

### 1-(2-Cyclopropylmethyl-2H-[1,2,4]triazol-3-yl)-ethanone

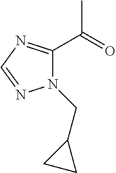

n-Butyl lithium (353 ml, 1.6M solution in THF, 0.565 moles) was added dropwise to a solution of 1-Cyclopropylmethyl-1H-[1,2,4]triazole (58 g, 0.47 moles) in THF (400 ml) at 0° C. After stirring for 1 hr at 0° C., N,N-dimethylacetamide (52.3 ml, 0.564 moles) was added to the reaction mixture and the stirring was continued for 1 hr. The progress of the reaction was monitored by TLC. The reaction mixture was quenched with sat. ammonium chloride solution and extracted with DCM. The combined organic layers were dried over sodium sulfate and concentrated. The crude material was purified by column chromatography (100% hexane followed by gradient elution to 10% ethyl acetate in hexane). Product (61 g) was obtained as light yellowish liquid. (Note: Since the product is volatile, it has to be distilled at less than 40° C.)

NMR (400 MHz, DMSO, δ): 0.36 (q, 2H), 0.46 (q, 2H), 1.25 (m, 1H), 2.48 (s, 3H), 4.33 (d, 2H), 8.12 (s, 1H).

MS (ES) (M+H)⁺: 166 for C₈H₁₁N₃O.

### Intermediate 78

### 3-(2-Cyclopropylmethyl-2H-[1,2,4]triazol-3-yl)-3-oxo-propionic acid ethyl ester

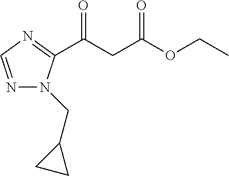

NaH (35.4 g, 1.476 moles of a 60% dispersion in oil) was added portionwise to a solution of 1-(2-Cyclopropylmethyl-2H-[1,2,4]triazol-3-yl)-ethanone (61 g, 0.369 moles) in 600 ml diethyl carbonate at 0° C. The mixture stirred at 0° C. for 1 hr and then heated to 60° C. for 2 hrs. The progress of the reaction was monitored by LCMS. After cooling to −10° C. by using ice-salt mixture, 6N HCl was added slowly to the reaction mixture by using additional funnel. The pH of the mixture was brought to about 7 with NaHCO₃ and extracted 4 times with EtOAc. The EtOAc was dried (Na₂SO₄) and concentrated to give oil that was chromatographed on silica gel (100% hexane followed by gradient elution to 40% EtOAc in hexane). Product (22.5 g) was obtained as a yellowish oily liquid.

NMR (400 MHz, DMSO, δ): 0.48 (q, 2H), 0.50 (q, 2H), 1.14 (t, 3H), 1.22 (m, 1H), 4.07 (q, 2H), 4.12 (s, 2H), 4.33 (d, 2H), 8.16 (s, 1H).

MS (ES) (M+H)⁺: 238 for C₁₁H₁₅N₃O₃.

### Intermediate 79

### 2-Amino-4-(2-cyclopropylmethyl-2H-[1,2,4]triazol-3-yl)-thiazole-5-carboxylic acid ethyl ester

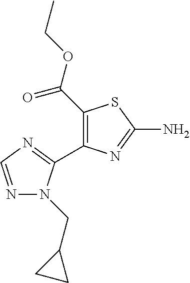

Sulfuryl chloride (9.0 ml, 0.112 moles) was added dropwise through the dropping funnel over a period of 30 min to a solution of 3-(2-Cyclopropylmethyl-2H-[1,2,4]triazol-3-yl)-3-oxo-propionic acid ethyl ester (22.5 g, 0.094 moles) in 1 L of DCM at 0° C. The reaction mixture was stirred for overnight at RT and quenched with aqueous sodium bicarbonate solution. The organic layer was separated and dried over sodium sulfate and concentrated. The residue was purified by silica gel column chromatography using 10% ethyl acetate in hexane. To a solution of above chloro compound in ethanol (500 ml), thiourea (8.58 g, 0.112 moles) was added and refluxed for 6 hrs. The progress of the reaction was monitored by TLC. After completion of the reaction, the reaction mixture was cooled to RT and concentrated under reduced pressure. Ice-cooled water was added to the residue and neutralized with saturated sodium carbonate solution (200 ml). The precipitated solid was filtered and washed with water, ether and dried under high vacuum to afford the product as white solid. (10 g).

NMR (400 MHz, DMSO, δ): 0.2 (q, 2H), 0.41 (q, 2H), 1.02 (m, 1H), 3.84 (d, 2H), 4.00 (q, 2H), 7.98 (s, 1H), 8.07 (bs, 2H).

MS (ES) (M+H)⁺: 294 for C₁₂H₁₅N₅O₂S.

### Intermediate 80

### 2-Chloro-4-(2-cyclopropylmethyl-2H-[1,2,4]triazol-3-yl)-thiazole-5-carboxylic acid ethyl ester

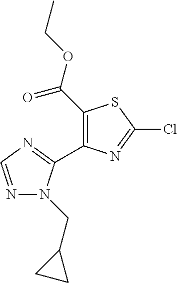

2-Amino-4-(2-cyclopropylmethyl-2H-[1,2,4]triazol-3-yl)-thiazole-5-carboxylic acid ethyl ester (10 g, 0.034 moles) was added to a mixture of acetic acid (100 ml) and conc. HCl (100 ml) at 0 to −5° C. Sodium nitrite (6.58 g, 0.095 moles) in water (100 ml) was added dropwise to above mixture. After complete addition, the reaction mixture was stirred for 1 hr at 0° C. Then urea (3.06 g, 0.51 moles) in water (100 ml) was added very slowly and stirring was continued at the same temperature for another 1 hr. The progress of the reaction was monitored by TLC. After completion of the reaction, ice cooled water was added, basified with sodium bicarbonate and extracted with ethyl acetate. The organic layer was separated, dried over sodium sulfate and concentrated. The residue was purified by silica gel column chromatography using 30% ethyl acetate in hexane to afford the product as light brown viscous oil. (7.5 g).

NMR (400 MHz, DMSO, δ): 0.43 (q, 2H), 0.45 (q, 2H), 1.14 (m, 1H), 3.96 (d, 2H), 4.18 (q, 2H), 8.10 (s, 1H).

MS (ES) (M+H)⁺: 313 for C₁₂H₁₃ClN₄O₂S.

### Intermediate 81

### 1-(2-Methoxy-ethyl)-1H-[1,2,4]triazole

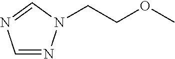

A solution of DBU (210.7 g, 1.737 moles) in THF (200 ml) was added dropwise to a mechanically stirred suspension of 1,2,4-triazole (100 g, 1.44 moles) and 2-Bromoethyl methyl ether (214.4 g, 1.737 moles) in dry THF (1 L) at 0° C., over a period of 3 hrs via addition funnel. The reaction mixture was allowed to warm to RT and stirred for overnight. The reaction mixture was filtered through celite and washed with THF (2×250 ml). The is combined filtrates were concentrated and the residue was distilled under reduced pressure to give the product as colorless liquid. (160 gm)

NMR (400 MHz, DMSO, δ): 3.21 (s, 3H), 3.66 (t, 2H), 4.32 (t, 2H), 7.95 (s, 1H), 8.45 (s, 1H).

### Intermediate 82

### 1-[2-(2-Methoxy-ethyl)-2H-[1,2,4]triazol-3-yl]-ethanone

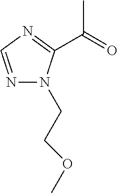

n-Butyl lithium (944 ml, 1.6M solution in THF, 1.51 moles) was added dropwise to a solution of 1-(2-methoxy-ethyl)-1H-[1,2,4]triazole (160 g, 1.26 moles) in THF (1 L) at −78° C. After stirring for 1 hr at −78° C., N,N-dimethylacetamide (131.6 ml, 1.51 moles) was added to the reaction mixture and the stirring was continued at the same temperature for 1 hr. The progress of the reaction was monitored by TLC. The reaction mixture was quenched with sat. ammonium chloride solution and extracted with DCM. The combined organic layers were dried over sodium sulfate and concentrated. The crude material was purified by column chromatography (100% hexane followed by gradient elution to 10% ethyl acetate in hexane). Product (100 g) was obtained as light yellowish liquid. NMR (400 MHz, CDCl3, δ): 2.72 (s, 3H), 3.31 (s, 3H), 3.76 (t, 2H), 4.78 (t, 2H), 7.95 (s, 1H).

MS (ES) (M+H)⁺: 170 for C₇H₁₁N₃O₂.

### Intermediate 83

### 3-[2-(2-Methoxy-ethyl)-2H-[1,2,4]triazol-3-yl]-3-oxo-propionic acid ethyl ester

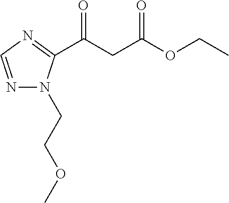

NaH (21.2 g, 0.887 moles of a 60% dispersion in oil) was added portionwise to a solution of 1-[2-(2-Methoxy-ethyl)-2H-[1,2,4]triazol-3-yl]-ethanone (100 g, 0.591 moles) in 1 L of diethyl carbonate at −50° C. The reaction mixture stirred at −50° C. for 1 hr and then it was allowed to warm to RT followed by heating at 60° C. for 2 hrs. The progress of the reaction was monitored by LCMS. After cooling to −10° C. by using ice-salt mixture, 6N HCl was added slowly to the reaction mixture by using additional funnel. The pH of the mixture was brought to about 7 with NaHCO₃ and extracted 4 times with EtOAc. The combined organic layer were dried over sodium sulfate and concentrated to give oil that was chromatographed on silica gel (100% hexane followed by gradient elution to 40% EtOAc in hexane). Product (35.6 g) was obtained as a yellowish oily liquid.

NMR (400 MHz, DMSO, δ): 1.16 (t, 3H), 3.17 (s, 3H), 3.68 (t, 2H), 4.07 (q, 2H), 4.11 (s, 2H), 4.67 (t, 2H), 8.17 (s, 1H).

MS (ES) (M+H)⁺: 242 for C₁₀H₁₅N₃O₄.

### Intermediate 84

### 2-Amino-4-[2-(2-methoxy-ethyl)-2H-[1,2,4]triazol-3-yl]-thiazole-5-carboxylic acid ethyl ester

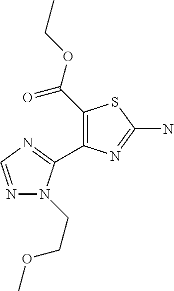

A solution of sulfuryl chloride (29.8 ml, 0.221 moles) in DCM (50 ml) was added dropwise through the dropping funnel over a period of 5 hr to a solution of 3-[2-(2-Methoxy-ethyl)-2H-[1,2,4]triazol-3-yl]-3-oxo-propionic acid ethyl ester (35.6 g, 0.147 moles) in DCM (400 ml) at 0° C. The reaction mixture was stirred for overnight at RT and quenched with aqueous sodium bicarbonate solution. The organic layer was separated, dried over sodium sulfate and concentrated. The residue was purified by silica gel column chromatography using 10% ethyl acetate in hexane to afford the product. (18 g).

To a solution of above chloro compound in ethanol (200 ml), thiourea (6.9 g, 0.091 moles) was added and refluxed for 6 hrs. The progress of the reaction was monitored by TLC. After completion of the reaction, the reaction mixture was cooled to RT and concentrated under reduced pressure. Ice-cooled water was added to the residue and neutralized with saturated sodium carbonate solution (200 ml). The precipitated solid was filtered and washed with water, ether and dried under high vacuum to afford the product as white solid. (12 g).

NMR (400 MHz, DMSO, δ): 1.03 (t, 3H), 3.08 (s, 3H), 3.59 (t, 2H), 4.01 (q, 2H), 4.12 (t, 2H), 7.99 (s, 1H), 8.06 (bs, 2H).

MS (ES) (M+H)⁺: 298 for C₁₁H₁₅N₅O₃S.

### Intermediate 85

### 2-Chloro-4-[2-(2-methoxy-ethyl)-2H-[1,2,4]triazol-3-yl]-thiazole-5-carboxylic acid ethyl ester

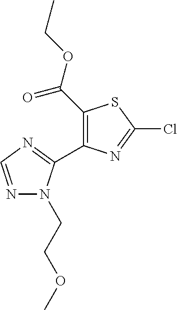

2-Amino-4-[2-(2-methoxy-ethyl)-2H-[1,2,4]triazol-3-yl]-thiazole-5-carboxylic acid ethyl ester (12 g, 0.04 moles) was added to a mixture of acetic acid (50 ml) and conc. HCl (50 ml) at 0 to −5° C. Sodium nitrite (7.8 g, 0.113 moles) in water (50 ml) was added dropwise to above mixture. After complete addition, the reaction mixture was stirred for 1 hr at 0° C. Then urea (3.6 g, 0.06 moles) in water (50 ml) was added very slowly and stirring was continued for another 1 hr. The progress of the reaction was monitored by TLC. After completion of the reaction, ice cooled water was added, basified with sodium bicarbonate and extracted with ethyl acetate. The organic layer was separated, dried over sodium sulfate and concentrated. The residue was purified by silica gel column chromatography using 30% ethyl acetate in hexane to afford the product (6.5 g).

NMR (400 MHz, DMSO, δ): 1.10 (t, 3H), 3.06 (s, 3H), 3.59 (t, 2H), 4.18 (q, 2H), 4.23 (t, 2H), 8.11 (s, 1H).

MS (ES) (M+H)⁺: 317 for C₁₁H₁₃ClN₄O₃S.

### Mycobacteria Susceptibility Testing Methods

Protocol for MIC testing: Microplate Alamar Blue Assay (Franzblau et al, 1998. J. Clin. Microbiol. 36: 362-366)

Two hundred microliters of sterile deionized water was added to all outer-perimeter wells of zo sterile 96-well plates to minimize evaporation of the medium in the test wells during incubation. Serial two-fold dilutions of the compounds in DMSO were made in another 96 well plate starting from 64 μg/ml to 0.5 μg/ml. 4 μl volumes of these were dispensed into the wells in rows B to G in columns 2 to 10 by using a multichannel pipette. 200 μl of M. tuberculosis culture diluted to a cell number of about 5×10⁵ cfu/ml was added to all the wells and the contents of the wells were mixed well. Three wells in column 11 served as drug-free (inoculum-only) controls. And 3 wells served as drug-free medium controls. The plates were incubated at 37 deg C. for 5 days. Fifty microliters of a freshly prepared 1:1 mixture of Alamar Blue (Accumed International, Westlake, Ohio) reagent and 10% Tween 80 was added to well B11. The plates were reincubated at 37° C. for 24 h. If well B11 turned pink, the reagent mixture was added to all wells in the microplate (if the well remained blue, the reagent mixture would be added to another control well and the result would be read on the following day). The microplates were re-incubated for an additional 24 h at 37° C., and the colors of all wells were recorded. A blue color in the well was interpreted as no growth, and a pink color was scored as growth.

The MIC was defined as the lowest drug concentration which prevented a color change from blue to pink

Compounds of the invention were tested in the above mentioned assay and the results are set out in the following table:

| Ex- | | Msmeg | | | | |
|---|---|---|---|---|---|---|
| am- | | IC50 | Mtu MIC | Hinf_MIC | Sau_MIC | Spn_MIC |
| ple | Structure | (uM) | (ug/ml) | (ug/ml) | (ug/ml) | (ug/ml) |
| 1 | 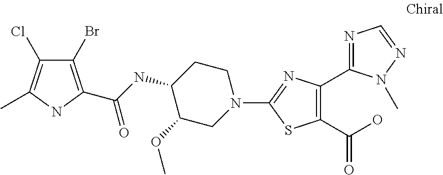 | 0.0026 | 0.008 | 8 | 0.116 | 0.116 |
| 2 | 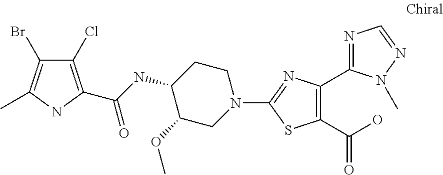 | <0.004 | <0.03 | 2.6 | 0.10 | 0.10 |
| 3 | 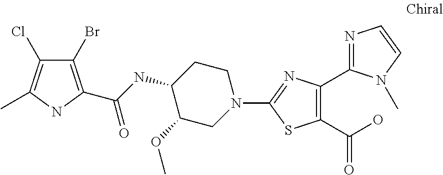 | 0.006 | 0.125 | 0.25 | 0.125 | 0.125 |
| 4 | 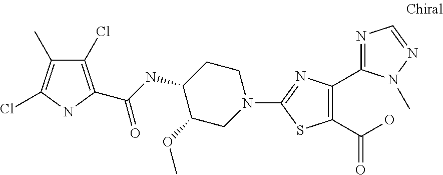 | 0.030 | 0.125 | 4 | 1.26 | 0.10 |
| 5 | 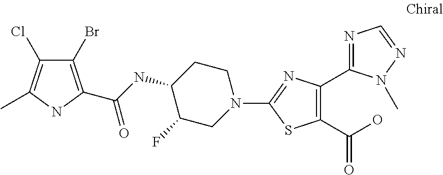 | <0.004 | <0.06 | 27.34 | 0.106 | 0.106 |
| 6 | 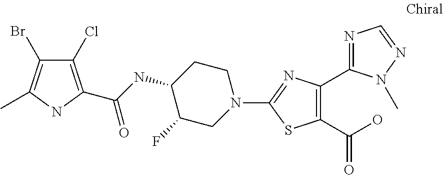 | 0.005 | 1 | 0.25 | 0.125 | 0.5 |
| 7 | 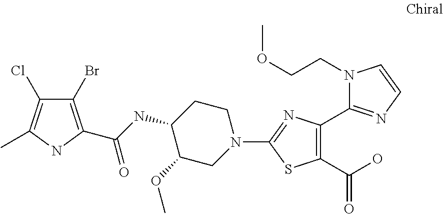 | 0.0046 | 1 | 0.25 | 0.125 | 0.125 |
| 8 | 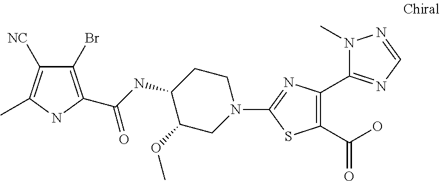 | <0.00025 | <0.03 | 2 | 0.5 | 0.125 |
| 9 | 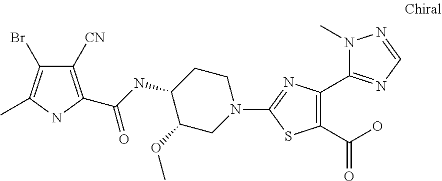 | 0.010 | 0.015 | 0.25 | 0.25 | 0.125 |
| 10 | 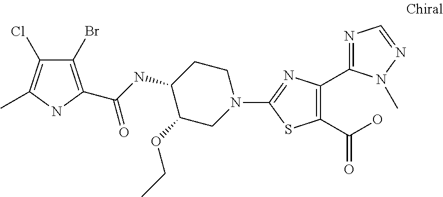 | <0.0025 | 0.008 | 0.125 | 0.125 | 0.125 |
| 11 | 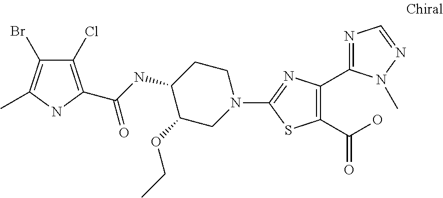 | <0.0025 | 0.06 | ND | 0.125 | 0.125 |
| 12 | 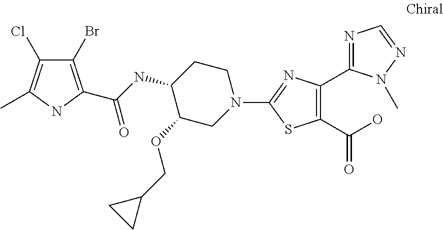 | 0.004 | 0.015 | 0.125 | 0.125 | 0.125 |
| 13 | 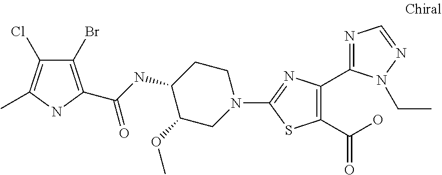 | <0.0025 | 0.015 | 0.125 | 0.125 | 0.125 |
| 14 | 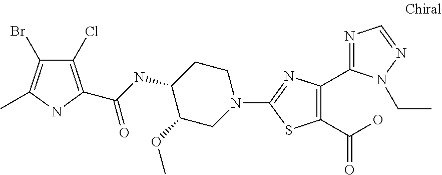 | <0.0025 | 0.03 | 0.125 | 0.125 | 0.125 |
| 15 | 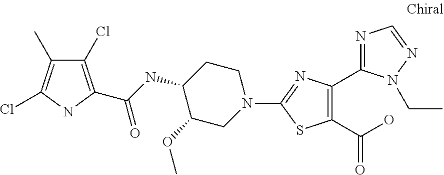 | 0.045 | 0.5 | 1 | 0.25 | 0.25 |
| 16 | 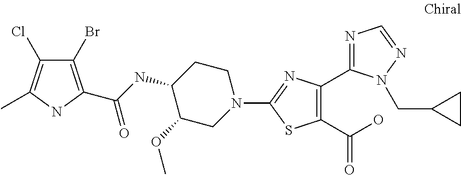 | <0.0025 | 0.03 | 0.25 | 8 | 0.25 |
| 17 | 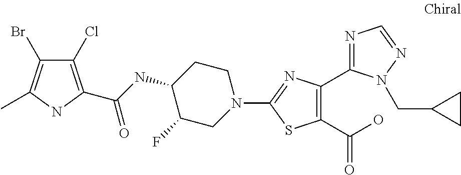 | 0.004 | 0.06 | 0.125 | 0.125 | 0.125 |
| 18 | 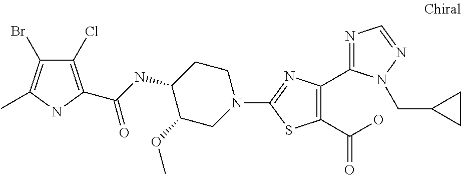 | 0.004 | 0.06 | 0.5 | 0.125 | 0.25 |
| 19 | 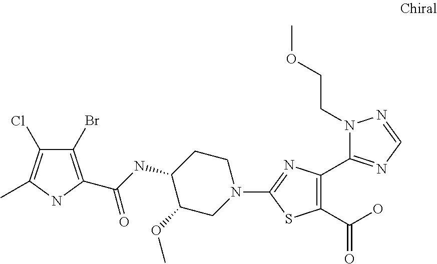 | <0.0025 | 0.03 | 0.25 | 0.125 | 0.125 |
| 20 | 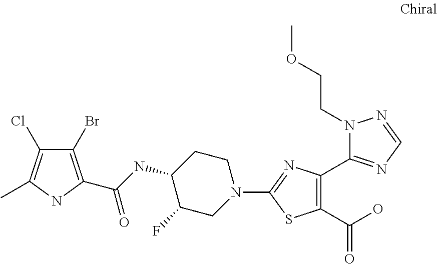 | 0.004 | 0.125 | 0.125 | 0.125 | 0.125 |
| 21 | 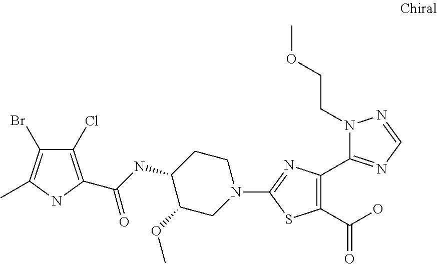 | <0.0025 | 0.06 | ND | 0.125 | 0.125 |
| 22 | 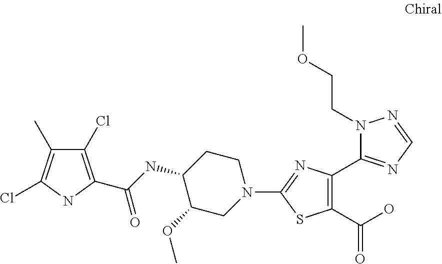 | 0.047 | 0.5 | ND | 0.25 | 0.5 |

1. A compound of formula (I): 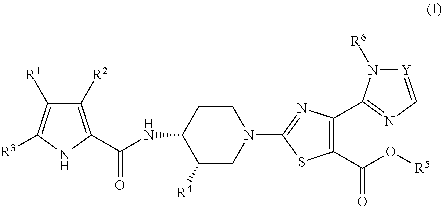
wherein:
(i) R¹ is Cl, R² is Br or CF₃ and R³ is CH₃,
(ii) R¹ is Br, R² is Cl, Br, CN or CF₃ and R³ is CH₃,
(iii) R¹ is CN, R² is Br or CF₃ and R³ is CH₃, or
(iv) R¹ is CH₃, R² and R³ are Cl;
R⁴ is H, fluoro, methyl, methoxy, ethoxy, cyclopropylmethoxy, propoxy, allyloxy and benzyloxy;
R⁵ is hydrogen or C₁₋₄alkyl;
Y=N or C—R^{a} wherein R^{a}=H, CH₃, F, CF₃, or CN;
R⁶ is selected from any one of C₁₋₄alkyl, C₁₋₄ haloalkyl C₂₋₄alkenyl, C₂₋₄alkenyl, C₃₋₆cycloalkyl, (C₃₋₆cycloalkyl)alkyl, (C₁₋₄alkoxy)alkyl, (C₃₋₆ cycloalkoxy)alkyl, (C₁₋₄ haloalkoxy)alkyl, C₁₋₄alkanoyl, N—(C₁₋₄alkyl)alkyl, N,N—(C₁₋₄alkyl)₂alkyl, carbocyclyl-R⁷— or heterocyclyl-R⁸—
R⁷, R⁸, are independently selected from a direct bond, —O—, —N(R⁹)—, —C(O)—, —N(R¹⁰)C(O)—, —C(O)N(R¹¹)—, —S(O)ₚ—, —SO₂N(R¹²)— or —N(R¹³)SO₂—; wherein R⁹, R¹⁰, R¹¹, R¹² and R¹³ are independently selected from hydrogen or C₁₋₄alkyl and p is 0-2;
and pharmaceutically acceptable salts thereof.

2. A compound of formula (I), or a pharmaceutically acceptable salt thereof, as claimed in claim 1, wherein:
a) R¹ is Cl, R² is Br, and R³ is CH₃;
b) R¹ is Br, R² is Cl or CN;
c) R¹ is CN, R² is Br, and R³ is CH₃; and
d) R¹ is CH₃, and R² and R³ are Cl.

3. A compound of formula (I), or a pharmaceutically acceptable salt thereof, as claimed in claim 1, wherein R⁴ is selected from fluoro, methoxy, ethoxy, and cyclopropylmethoxy.

4. A compound of formula (I), or a pharmaceutically acceptable salt thereof, as claimed in claim 1, wherein Y is selected from CH and N.

5. A compound of formula (I), or a pharmaceutically acceptable salt thereof, as claimed in claim 1, wherein R⁶ is selected from C₁₋₄ alkyl, (C₁₋₄ alkoxy)alkyl, and (C₃₋₆ cycloalkyl)alkyl.

6. A compound of formula (I), or a pharmaceutically acceptable salt thereof, as claimed in claim 1 for use as a medicament.

7. A compound of formula (I), or a pharmaceutically acceptable salt thereof, as claimed in claim 1, for use in therapy.

8. A compound of formula (I), or a pharmaceutically acceptable salt thereof, as claimed in claim 1, for use in the treatment of a mycobacterial infection.

9. (canceled)

10. A method for inhibition of bacterial DNA gyrase and/or topoisomerase IV in a warm-blooded animal, such as a human being, in need of such treatment, said method comprising administering to said animal an effective amount of a compound of formula (I) or a pharmaceutically acceptable salt thereof, as claimed in claim 1.

11. A compound of the formula (I), or a pharmaceutically acceptable salt thereof, as claimed in claim 1, and a chemotherapeutic agent selected from:
i) one or more additional antibacterial agents; and/or
ii) one or more anti-infective agents; and/or
iii) biological protein therapeutics for example antibodies, cytokines, bactericidal/permeability-increasing protein (BPI) products;
iv) one or more antibacterial agents useful in the treatment of Mycobacterium tuberculosis and/or
v) one or more efflux pump inhibitors.

12. A process for preparing a compound of formula (I) or a pharmaceutically acceptable salt thereof as claimed in claim 1 (wherein R¹, R², R³, R⁴, R⁵ and R⁶ are as defined in relation to formula (I)), which process comprises:
(a) reacting a compound of formula (II): 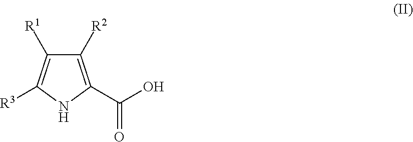
or an activated acid derivative thereof; with a compound of formula (III): 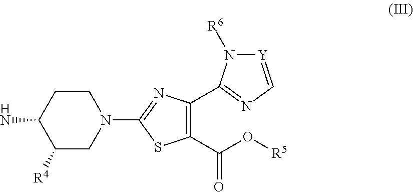
or
(b) reacting a compound of formula (IV) 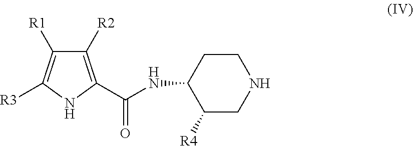
with a compound of formula (V): 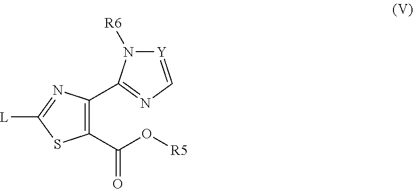
wherein L is a displaceable group; or
(c) for compounds of formula (I) wherein R⁵ is C₁₋₄alkyl; reacting a compound of formula (I) which is a compound of formula (VI) 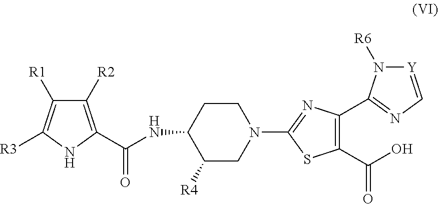
with a compound of formula (VII):
R^{4a}—OH (VII)
wherein R^{4a} is C₁₋₄alkyl;
or
(d) for compounds of formula (I) or wherein R⁵ is hydrogen; deprotecting a compound of formula (VIII) 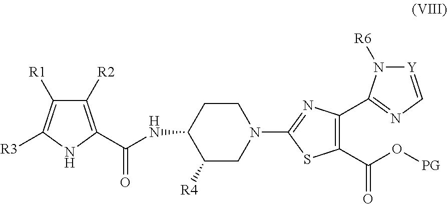
wherein PG is a carboxylic acid protecting group;
and thereafter if necessary:
i) converting a compound of the formula (I) into another compound of the formula (I);
ii) removing any protecting groups;
iii) forming a pharmaceutically acceptable salt; and/or
iv) chirally purifying the compound of formula (I).
